(19) United States
(12) Patent Application Publication
(10) Pub. No.: US 2025/0099350 A1
(43) Pub. Date: Mar. 27, 2025

(54) HAIR TREATMENT METHODS FOR SMOOTHING HAIR

(71) Applicant: L'OREAL, Paris (FR)

(72) Inventors: Lisa YE-TSE, Brooklyn, NY (US); Heather LEE, Wayne, NJ (US); Vibha SHAH, Branchburg, NJ (US); Ayano TAKEDA, Kawasaki (JP)

(73) Assignee: L'OREAL, Paris (FR)

(21) Appl. No.: 18/224,119

(22) Filed: Jul. 20, 2023

(51) Int. Cl. A61K 8/365; A45D 7/04; A61K 8/49; A61K 8/898; A61Q 5/06

(52) U.S. Cl. CPC A61K 8/365; A45D 7/04; A61K 8/4973; A61K 8/898; A61Q 5/06; A61K 2800/48; A61K 2800/884

(57) ABSTRACT

The instant disclosure is drawn to a hair treatment method including: (i) applying a hair smoothing composition including at least one keto acid to the hair, allowing the composition to remain on the hair, and rinsing the hair smoothing composition from the hair; (ii) applying a conditioning composition to the hair, allowing the conditioning composition to remain on the hair, and rinsing the conditioning composition from the hair; the conditioning composition including: one or more polar fatty compounds; one or more water-miscible solvents; and one or more cationic surfactants; wherein the conditioning composition is substantially free of water; and (iii) applying a leave-on finishing composition to the hair, the leave-on finishing composition including: one or more cyclic carbonates, one or more silicones; and water.

### FIELD OF THE DISCLOSURE

The present disclosure relates to hair treatment methods of treating hair with the compositions. Treatment with the compositions provide hair with improved fiber alignment, frizz control, and smoothness.

### BACKGROUND

Many consumers use cosmetic and care compositions to enhance the appearance of hair, e.g., by changing the color, style, or shape of the hair and/or by imparting various cosmetic properties to hair, such as shine and conditioning. Hair can become dry or damaged for various reasons, e.g., weather exposure, poor nutrition, mechanical treatments (e.g., brushing hair), styling treatments using chemicals, dying, heat, nutrition, etc. Even cleansing products can remove hair's natural oils causing dryness, which can lead to a dull appearance, split ends, and frizz.

Chemical treatments for hair include bleaching and coloring treatments to change the color the hair. Chemical treatments also include processes to permanently change the shape and structure of the hair, for example by perming, waving, relaxing or straightening the hair. These chemical treatments change the look of hair by changing its physical structure, which inevitably causes a certain degree of damage to the hair. Environmental factors, such as salt water, sunlight, and heat, are also known to damage hair. Damaged hair is characterized by unnatural changes to the protein structure of the individual hair strands or shafts.

The popularity and usage of oils for hair treatments has increased due to their effectiveness and simplicity. Commonly used oils include olive oil, mineral oil, avocado oil, apricot kernel oil, rice bran oil, and coconut oil. However, these treatments can leave the hair feeling greasy. In addition, the effects are not usually seen after more than several hours (e.g., 8 hours) of treatment and several treatments are usually required, making it time consuming and labor intensive.

Damage to hair results in split ends, dryness, hair that is easily broken, and hair that becomes “frizzy” and unmanageable. Because the visible portion of hair is dead, it has no ability to regenerate itself. There are numerous over the counter and salon treatments that purport to repair damaged hair. These include conditioners, hot oil treatments, hydrolyzed proteins, vitamin formulations, and exotic fruit, leaf, or root extracts. These treatments, however, provide only limited improvement to the hair. Therefore, hair treatment technologies that can straighten, relax, or style the hair without chemically damaging the hair are desired.

There is still a need for providing improved manageability of hair, for example, improved hair alignment, reduced unwanted volume (especially reduced frizz), make hair smoother and increase shine.

### SUMMARY OF THE DISCLOSURE

The instant disclosure is drawn to methods for improving the look, feel, and style of hair. In particular, the hair treatment compositions and methods improve fiber alignment, reduce frizz, and impart smoothness. The treated hair is soft, shiny, conditioned, and has a healthy appearance. The methods form a smooth texture on the surface of the hair, which is surprisingly durable. The desirable cosmetic properties imparted to the hair are long-lasting, resistant to humidity, and maintained even washing the hair.

The methods of the instant disclosure typically include:
(i) applying a hair smoothing composition comprising at least one keto acid optionally having pH of about 2 to about 4 to the hair, allowing the composition to remain on the hair, and rinsing the hair smoothing composition from the hair;
(ii) applying a conditioning composition to the hair, allowing the conditioning composition to remain on the hair, and rinsing the conditioning composition from the hair; the conditioning composition comprising:
   (a) one or more polar fatty compounds;
   (b) one or more water-miscible solvents; and
   (c) one or more cationic surfactants; wherein the conditioning composition is substantially free of water; and
(iii) applying a leave-on finishing composition to the hair, the leave-on finishing composition comprising:
   (a) one or more cyclic carbonates
   (b) one or more silicones; and
   (c) water.

After applying the leave-on finishing composition, the hair can be dried, for example, with a blow dryer such as, for example, to be essentially completely dry. This can be done without rinsing the leave-on finishing composition from the hair first. Subsequently, the hair can be treated with a thermal heat treatment such as one occurring at a temperature of about 150 to about 280° C. In various embodiments, the heat treatment occurs at a temperature of about 150 to about 250° C., 150 to about 240° C. about 180 to about 300° C., about 180 to about 280° C., about 200 to about 300° C., about 200 to about 280° C., about 200 to about 260° C., about 200 to about 250° C., about 210 to about 300° C., about 210 to about 280° C., about, 180° C., about 190° C., about 200° C., about 210° C., about 220° C., about 230° C., about 240° C., about 250° C. or about 260° C.

In various embodiments, the heat treatment includes treating (i.e., drying) the hair with a hot iron, for example, a curling iron or a flat iron. The hot iron can be passed over the hair once or multiple times, for example, one or more passes, two or more passes, three or more passes, or four or more passes.

Keto acids useful in the hair smoothing composition are compounds that include both at least one carboxylic acid group as well as at least one ketone group. Nonlimiting examples include those keto acids having two to five carbon atoms (C2-C5) such as glyoxylic acid and levulinic acid.

Polar fatty compounds useful in the conditioning composition include fatty alcohols, fatty acids, and fatty esters. The polar fatty compound may have one or more C8 or higher carbon chains.

Cationic surfactants useful in the conditioning composition include fatty amidoamines, quarternized amines and the like.

Water-miscible solvents useful in the conditioning composition include polyols such as glycerin, C₁-C₆ mono-alcohols, glycols, ketal/acetal of glycerin compounds, and mixtures thereof. In various embodiments, at least one of the one or more water soluble solvents is glycerin, a glycol (e.g., ethylene glycol, propylene glycol, butylene glycol, pentylene glycol, caprylyl glycol, etc.) or a combination thereof.

Cyclic carbonates useful in the leave-on finishing composition are also known as cyclic carbonate esters. Nonlimiting examples of cyclic carbonates include propylene carbonate, dipropylene carbonate, butylene carbonate, 2,3-butylene carbonate, 2,3-pentylene carbonate, pentylene carbonate, ethylene carbonate, glycerol carbonate, or a mixture thereof. In various embodiments, propylene carbonate is preferred.

Silicones useful in the leave-on finishing composition include silicone oils such as dimethicones and dimethiconols; and amino-functionalized silicones such as amodimethicone, bis-hydroxy/methoxy amodimethicone, bis-cetearyl amodimethicone, bis(C13-15 alkoxy) PG amodimethicone, aminopropyl phenyl trimethicone, aminopropyl dimethicone, bis-amino PEG/PPG-41/3 aminoethyl PG-propyl dimethicone, or a mixture thereof. In various embodiments, amodimethicone is preferred.

The methods are useful for improving the look, feel, and style of hair. Accordingly, the instant disclosure is drawn to methods for treating hair, for example, methods for improving the look, feel, or style of the hair. More specifically, the instant disclosure is drawn to methods for improving fiber alignment, reducing frizz, and imparting smoothness to hair. Such methods typically include application of the hair smoothing composition, the conditioning composition, and the leave-on finishing composition to the hair. In certain embodiments, without rinsing the composition from the hair, the hair is dried and treated with a hot iron. In various embodiments, the temperature of the hot iron is at least 100° C., such as at least 150° C., for example, about 150° C. to about 240° C. The treated hair is soft, smooth, shiny, conditioned, not crunchy and with a healthy appearance. The smooth coating is surprisingly durable. The improved cosmetic properties imparted to the hair are long-lasting and are maintained even after washing the hair.

In various embodiments, the compositions of the instant disclosure can be used in a routine with one or more additional hair treatment steps. For example, a cleansing composition (shampoo) may be used, for example, prior to using the hair smoothing composition.

### BRIEF DESCRIPTION OF THE DRAWINGS

Implementation of the present technology is described, by way of example only, with reference to the attached FIGURE, wherein:

FIG. 1 shows hair swatches treated using methods of the instant disclosure, comparative methods, and with baseline swatch.

It should be understood that the various aspects are not limited to the arrangements and instrumentality shown in the drawings.

### DETAILED DESCRIPTION OF THE DISCLOSURE

The instant disclosure is drawn to methods for treating hair, such as hair in need of reduced frizz and/or improved smoothness. This is achieved using a sequence of composition(s) that includes a unique combinations of ingredients: typically using a low pH, keto acid-containing hair smoothing composition; an anhydrous conditioner including a polar fatty compound, cationic surfactant, and water-miscible solvent; and a leave-on aqueous finishing composition including cyclic carbonate, silicone and water.

The inventors believe that cyclic carbonates undergo a reaction when subjected to a thermal heat treatment. Heat activates a reaction with the (propylene) carbonate and amine groups of the hair fibers, likely forming an N,N′-disubstituted urea linkage that contributes to the long-lasting fixation of the newly formed shape.

The instant methods are gentle, long-lasting, and reduce hair frizz of while simultaneously improving smoothness. The methods improve fiber alignment, reduce frizz, and impart smoothness, to a surprising degree. The longevity of the improved fiber alignment, reduced frizz, and smoothness is also significant and surprising. Hair treated in accordance with the methods outline herein retains these desirable cosmetic properties, even after multiple cleansing cycles. Thus, the benefits provided by the disclosed methods are surprisingly long-lasting and wash resistant.

Methods in accordance with the instant disclosure typically include:
(ii) applying a hair smoothing composition comprising at least one keto acid and optionally having pH of about 2.0 to about 4.0 to the hair, allowing the composition to remain on the hair, and rinsing the hair smoothing composition from the hair;
(ii) applying a conditioning composition to the hair and rinsing the conditioning composition from the hair; the conditioning composition comprising:
   (a) one or more polar fatty compounds;
   (b) one or more water-miscible solvents; and
   (c) one or more cationic surfactants; wherein the conditioning composition is substantially free of water; and
(iii) applying a leave-on finishing composition to the hair; the leave-on finishing composition comprising:
   (a) one or more cyclic carbonates
   (b) one or more silicones; and
   (c) water.

After applying the leave-on composition, the hair can be dried, for example, with a blow dryer or can dry naturally. Subsequently, the hair is desirably treated with a thermal heat treatment. The thermal heat treatment typically occurs at a temperature of about 150 to about 240° C. In various embodiments, the heat treatment occurs at a temperature that is preferably 150° C. to about 240° C., more preferably about 180° C. to about 230° C.

In various embodiments, the heat treatment includes treating the hair with a hot iron, for example, a curling iron or a flat iron. The hot iron can be passed over the hair once or multiple times, for example, one or more passes, two or more passes, three or more passes, or four or more passes.

### Methods

The hair treatment compositions disclosed herein are particularly useful in methods for treating hair, preferably human hair, in particular human hair of the head. Treatment with the compositions improves fiber alignment, reduces frizz, and impart smoothness to the hair. The methods typically comprise applying a hair smoothing composition to the hair. Prior to treating the hair with the hair smoothing composition, the hair may be shampooed such as with a conventional shampoo, e.g., one that is sulfate free, and rinsed to remove residual shampoo from the hair. The hair should preferably be wet or damp. In other embodiments the hair may be treated with a relaxer (alkaline treatment) prior to treating with the hair smoothing composition and/or prior to treatment with the optional shampoo treatment. In any case, the hair is preferably wet or damp at the time of treatment with the hair smoothing composition.

The hair is treated with a hair smoothing composition that includes at least one keto acid. Keto acid in the hair smoothing composition, such as glyoxylic acid provides smoothing to the hair fibers without breaking the cystine disulfide bridge. Therefore, it provides long-lasting relaxing effects without causing damage to the hair and scalp, which is more common with alkaline chemical agents. The carboxylic and aldehyde groups of glyoxylic acid react with the amine groups in hair keratin resulting in the formation of stable bonds. The hair smoothing composition including keto acid typically have an acidic pH, such as from about 2.5 to about 5. In various embodiments, the pH of the hair smoothing composition is about 2.0 to about 4.0 or 2.5 to about 4.5, about 2.5 to about 4, about 2.5 to about 3.5, or about 2.8 to about 3.2. The hair smoothing compositions may be applied to the hair, allowed to remain or dwell on the hair for a period of time (e.g., 1 to 30 min., 1 to 20 min., 1 to 10 min., 5 to 30 min., 5 to 25 min., 5 to 20 min., 5 to 15 min., 10 to 30 min., 10 to 25 min., 10 to 20 min., or 10 to 15 min.), and then rinsed from the hair.

After rinsing the hair smoothing composition from the hair and optionally towel-drying the hair, the hair is then treated with a conditioning composition that includes (a) a polar fatty compound; (b) a water-miscible solvent; and (c) cationic surfactant. The conditioning composition is anhydrous, i.e., substantially free of water. The conditioning composition may be applied to the hair, allowed to remain on the hair for a period of time (e.g., 1 to 30 min., 1 to 20 min., 1 to 10 min., 5 to 30 min., 5 to 25 min., 5 to 20 min., 5 to 15 min., 10 to 30 min., 10 to 25 min., 10 to 20 min., or 10 to 15 min.), and then rinsed from the hair. Alternatively, the conditioning composition may be applied to the hair, massaged into the hair thoroughly and rinsed with no appreciable dwell time, and optionally towel-dried.

After rinsing the conditioning composition and optional towel drying, the hair is then

treated with a leave-on finishing composition that includes (a) one or more cyclic carbonates, (b) silicone; and (c) water.

In various embodiments, the pH of the leave-on finishing composition is about 2 to about 6, about 2.5 to about 5.5 or 2.5 to about 5.0, about 2.5 to about 4.5, about 2.0 to about 5.0, about 3.0 to about 5.0, or about 3.0 to about 4.5, or about 3.5 to about 4.5. The leave-on finishing composition may be applied to the hair, allowed to remain on the hair for a period of time (e.g., 1 to 30 min., 1 to 20 min., 1 to 10 min., 5 to 30 min., 5 to 25 min., 5 to 20 min., 5 to 15 min., 10 to 30 min., 10 to 25 min., 10 to 20 min., or 10 to 15 min.), and then rinsed from the hair. Again, alternatively, no appreciable dwell time may be used.

Application of the leave-on finishing composition to the hair will moisten the hair because the compositions are aqueous (contain a substantial amount of water). Therefore, after massaging or spreading the composition throughout the hair, without rinsing the composition from the hair, the hair can be dried, for example, the hair can be dried using a blow dryer. After the hair is dry, the hair is then preferably treated with a thermal treatment (treated with heat). For example, the hair can be treated with a hot iron, in particular, a flat iron. Typically, the hot iron is passed over the hair at least once, at least twice, at least three times, or more.

The hair may be dried and treated with a hot iron, such as a flat iron, at a temperature of about 150° C. to about 240° C., preferably 150° C. to about 240° C., more preferably about 180° C. to about 230° C.

The treated hair is also soft, shiny, conditioned, with a healthy appearance. The compositions include a unique combination of a cyclic carbonate and a(n amino-functionalized) silicone, which forms a smooth coating on the surface of the hair. The smooth coating is surprisingly durable. The improved cosmetic properties imparted to the hair are long-lasting and are maintained even after multiple washing cycles.

### Compositions

The compositions useful in methods of the present invention interact with one another in a synergistic-like manner. Treatment with the compositions improve fiber alignment, reduce frizz, and impart smoothness, to a surprising degree. The longevity of the improved fiber alignment, reduced frizz, and smoothness is also significant and surprising. Hair treated with the compositions of the instant disclosure retains these desirable cosmetic properties, even after multiple cleansing cycles. Thus, the benefits provided by the compositions are long-lasting and wash resistant.

### Hair Smoothing Composition

The hair smoothing composition useful in the present invention includes at least one keto acid. By keto acid, it is meant a compound that includes both at least one carboxylic acid group (—COOH) as well as at least one ketone group (—C═O). Keto acids generally provide semi-permanent hair smoothing without breaking the cystine disulfide bridge. They can provide long lasting smoothing effects to hair fibers, without causing damage to the hair and scalp irritations.

Nonlimiting examples of keto acids include those having two to five carbon atoms. Examples of such C2-C5 keto acids include glyoxylic acid (C₂H₂O₃) and levulinic acid (C₅H₈O₃). The keto acid may have a pKa from about 3.0 to about 5.0. These compounds may, for example, be introduced into the composition as an acid and/or as salt.

The total amount of the one or more keto acids in the hair smoothing composition, if present, will vary. Nonetheless, in various embodiments, the compositions include about 0.1 to about 20 wt. % of the one or more keto acids, based on the total weight of the compositions. In further embodiments, the compositions include about 0.5 to about 15 wt. %, about 0.5 to about 12 wt. %, about 0.5 to about 10 wt. %, about 1 to about 20 wt. %, about 1 to about 15 wt. %, about 1 to about 10 wt. %, about 2 to about 20 wt. %, about 2 to about 15 wt. %, about 2 to about 10 wt. %, about 5 to about 20 wt. %, about 5 to about 15 wt. %, about 5 to about 10 wt. %, based on the total weight of the compositions. According to certain embodiments, the hair smoothing composition includes about 1 to about 10% glyoxylic acid and about 1 to about 5% levulinic acid.

The hair smoothing composition may have a pH adjusted to be within a range from about 2.0 to about 5.0, about 2.0 to about 4.0 or 2.5 to about 4.5, about 2.5 to about 4, about 2.5 to about 3.5, or about 2.8 to about 3.2. Accordingly, it is desirable for the hair smoothing composition to be aqueous. According to certain embodiments, the hair smoothing composition has between about 35% to about 90% water, such as from about 40% or 45% to about 80% water.

Other ingredients may be included in the hair smoothing composition such as one or more water-miscible solvents, one or more surfactants or emulsifiers, one or more non-silicone fatty compounds (e.g. fatty alcohols, fatty esters), and one or more miscellaneous ingredients. Water-miscible solvents, surfactants or emulsifiers, non-silicone fatty compounds, and miscellaneous ingredients are described within this specification in the section, “Leave-on Finishing Composition.” For water-miscible solvents, polyols and monoalcohols are notable. For non-silicone fatty compounds, fatty alcohols and fatty esters are notable. For surfactants and emulsifiers, alkyl and polyalkyl ethers of poly(ethylene oxide) are notable.

The total amount of the one or more water miscible solvents in the hair smoothing compositions, if present, will vary. Nonetheless, in various embodiments, the hair smoothing compositions include about 0.1 to about 20 wt. % of the one or more water soluble solvents, based on the total weight of the compositions. In further embodiments, the hair smoothing compositions include about 0.1 to about 15 wt. %, about 0.1 to about 10 wt. %, about 0.5 to about 20 wt. %, about 0.5 to about 15 wt. %, about 0.5 to about 10 wt. %, about 1 to about 20 wt. %, about 1 to about 15 wt. %, about 1 to about 10 wt. %, about 2 to about 20 wt. %, about 2 to about 15 wt. %, about 2 to about 10 wt. %, about 5 to about 20 wt. %, about 5 to about 15 wt. %, or about 5 to about 10 wt. %, based on the total weight of the compositions.

The total amount of the surfactants or emulsifiers in the hair smoothing composition, if present, will vary. Nonetheless, in various embodiments, the compositions include about 0.1 to about 10 wt. % of the one or more nonionic surfactants or emulsifiers. In further embodiments, the compositions include about 0.1 to about 8 wt. %, about 0.1 to about 5 wt. %, about 0.1 to about 1 wt. %, about 0.5 to about 10 wt. %, about 0.5 to about 8 wt. %, about 0.5 to about 5 wt. %, about 0.5 to about 3 wt. %, about 1 to about 10 wt. %, about 1 to about 8 wt. %, about 1 to about 5 wt. %, or about 1 to about 3 wt. %, based on the total weight of the compositions. In certain embodiments, the surfactants or emulsifiers are nonionic.

The total amount of the one or more non-silicone-based fatty compounds in the hair smoothing composition, if present, will vary. Nonetheless, in various embodiments, the compositions include about 0.1 to about 20 wt. % of the one or more non-silicone-based fatty compounds, based on the total weight of the compositions. In further embodiments, the compositions include about 0.1 to about 15 wt. %, about 0.1 to about 12 wt. %, about 0.1 to about 10 wt. %, about 0.1 to about 8 wt. %, about 0.1 to about 5 wt. %, about 0.5 to about 20 wt. %, about 0.5 to about 15 wt. %, about 0.5 to about 12 wt. %, about 0.5 to about 10 wt. %, about 0.5 to about 8 wt. %, about 0.5 to about 5 wt. %, about 1 to about 20 wt. %, about 1 to about 15 wt. %, about 1 to about 12 wt. %, about 1 to about 10 wt. %, about 1 to about 8 wt. %, about 1 to about 5 wt. %, about 2 to about 20 wt. %, about 2 to about 15 wt. %, about 2 to about 12 wt. %, about 2 to about 10 wt. %, about 2 to about 8 wt. %, about 2 to about 5 wt. %, or about 3 to about 5 wt. %, based on the total weight of the compositions.

The hair smoothing composition generally also includes water, such as from about 40 to about 80 wt. %. The hair smoothing composition may also include one or more of various miscellaneous ingredients. The total amount of the one or more miscellaneous ingredients in the hair smoothing composition, if present, will vary. Nonetheless, in various embodiments, the compositions include about 0.1 to about 15 wt. % of the one or more miscellaneous ingredients, based on the total weight of the compositions. In further embodiments, the compositions include about 0.1 to about 12 wt. %, about 0.1 to about 10 wt. %, about 0.1 to about 5 wt. %, about 0.5 to about 15 wt. %, about 0.5 to about 12 wt. %, about 0.5 to about 10 wt. %, about 0.5 to about 8 wt. %, about 0.5 to about 5 wt. %, about 1 to about 15 wt. %, about 1 to about 12 wt. %, about 1 to about 10 wt. %, about 1 to about 8 wt. %, about 1 to about 5 wt. %, about 2 to about 15 wt. %, about 2 to about 12 wt. %, about 2 to about 10 wt. %, about 2 to about 8 wt. %, or about 2 to about 5 wt. %, based on the total weight of the compositions.

The hair smoothing composition typically has a pH from about 2 to about 4 and may include:
(a) about 5 to 20 wt. % of one or more keto acids;
(b) about 35% to about 90% of water;
(c) optionally about 0.5 to about 10 wt. % of one or more water-miscible solvents;
(d) optionally about 0.1 to about 5 wt. % of at least one surfactant; and wherein all weight percentages are based on a total weight of the hair smoothing composition.

### Conditioning Composition

The conditioning compositions useful in the present invention include
(a) a polar fatty compound; (b) water-miscible solvent; and (c) cationic surfactant. Furthermore the conditioning composition is substantially free of water.

The term “cationic surfactant” means a surfactant that may be positively charged when it is contained in the compositions according to the disclosure. This surfactant may bear one or more positive permanent charges or may contain one or more functional groups that are cationizable in the composition according to the disclosure. Cationic surfactants useful in the conditioning composition include fatty amidoamines, quarternized amines and the like. Amidoamines are particularly notable in the hair smoothing composition.

Non-limiting examples of cationic surfactants include cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline; amidoamines such as stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, brassicamidopropyldimethylamine, lauramidopropyl dimethylamine, myristamidopropyl dimethylamine, dilinoleamidopropyl dimethylamine, palmitamidopropyl dimethylamine, and mixtures thereof.

The total amount of cationic surfactant(s) in the composition can vary but is typically from about 0.1 to about 10 wt. %, based on the total weight of the composition. In some cases, the total amount of cationic surfactant(s) is from about 0.1 to about 4 wt. %, about 0.1 to about 3 wt. %, about 0.1 to about 2 wt. %, about 0.5 to about 10 wt. %, about 0.5 to about 5 wt. %, about 0.5 to about 3 wt. %, based on the total weight of the composition.

Water-miscible solvents are described below with respect to leave-on finishing compositions. Similar water-miscible solvents are suitable for use in the conditioning composition. Polyols and monoalcohols are particularly notable. The total amount of the one or more water soluble solvents in the hair smoothing compositions, if present, will vary. Nonetheless, in various embodiments, the compositions include about 50 wt. % to about 98 wt. % of the one or more water soluble solvents, based on the total weight of the compositions. In further embodiments, the compositions include about 50 to about 98 wt. % to about 50 to about 95 wt. %, about 65 to about 98 wt. %, about 65 to about 98 wt. %, about 75 to about 98 wt. %, or about 75 to about 96 wt. %, based on the total weight of the compositions.

Polar fatty compounds useful in the conditioning composition are fatty compounds that include at least one polar functional group. The polar fatty compounds may be non-silicone-based fatty compounds. The polar fatty compounds may have one or more carbon chains of C8 or greater. The one or more carbon chains may be each independently greater than 8 carbon atoms, 8 to 50 carbon atoms, 8 to 40 carbon atoms, 8 to 30 carbon atoms, 8 to 22 carbon atoms, 12 to 22 carbon atoms, or 12 to 18 carbon atoms.

Non-limiting examples of polar fatty compounds include fatty esters, fatty alcohols, glyceryl esters (glycerol esters), fatty acids, fatty esters, alkyl ethers of fatty alcohols, fatty acid esters of fatty alcohols, fatty acid esters of alkyl ethers of fatty alcohols, fatty acid esters of alkoxylated fatty alcohols, fatty acid esters of alkyl ethers of alkoxylated fatty alcohols, hydroxy-substituted fatty acids, and mixtures thereof. Non-limiting examples of fatty esters include glycerol fatty esters, sucrose fatty esters, sorbitan fatty ester, fatty acid esters, or mixtures thereof. Non-limiting examples of the fatty alcohols, fatty acids, fatty alcohol derivatives, and fatty acid derivatives are found in International Cosmetic Ingredient Dictionary, Sixteenth Edition, 2016, which are incorporated by reference herein in its entirety. A more exhaustive but non-limiting list of useful polar fatty compounds are the polar variants listed under the heading “Non-silicone-based fatty compounds.” Particularly suitable examples include fatty alcohols (e.g. cetyl alcohol), fatty acids (e.g. oleic acid), and fatty esters.

The total amount of the one or more polar fatty compounds in the conditioning composition, if present, will vary. Nonetheless, in various embodiments, the compositions include about 0.1 to about 20 wt. %, of the one or more polar fatty compounds based on the total weight of the composition. The total amount of one or more polar fatty compounds may be from about 0.1 to about 15 wt. %, about 0.1 to about 10 wt. %, about 0.1 to about 8 wt. %, about 0.1 to about 5 wt. %, about 0.2 to about 5 wt. %, about 0.5 to about 20 wt. %, about 0.5 to about 15 wt. %, about 0.5 to about 10 wt. %, about 0.5 to about 8 wt. %, about 0.5 to 5 wt. %, about 1 to about 20 wt. %, about to about 15 wt. %, about 1 to about 10 wt. %, about 1 to about 8 wt. %, or about 1 to about 5 wt. %, including ranges and sub-ranges therebetween, based on the total weight of the composition.

The conditioning composition is anhydrous and may include:
(a) about 0.5 to 5 wt. % of at least one polar fatty compound;
(b) about 50 to about 95 wt. % of at least one water-miscible solvent; and
(c) about 0.5 to about 5 wt. % of at least one cationic surfactant, wherein all weight percentages are based on a total weight of the conditioning composition.

### Leave-On Finishing Composition

The leave-on finishing composition includes (a) one or more cyclic Carbonates; (b) one or more silicones; and (c) water.

### (a) Cyclic Carbonates

Cyclic carbonates are also known as cyclic carbonate esters. Nonlimiting examples of cyclic carbonates include propylene carbonate, dipropylene carbonate, butylene carbonate, 2,3-butylene carbonate, 2,3-pentylene carbonate, pentylene carbonate, ethylene carbonate, glycerol carbonate, or a mixture thereof. In various embodiments, propylene carbonate is preferred.

The total amount of the one or more cyclic carbonates in the compositions will vary. Nonetheless, in various embodiments, the compositions include about 1 to about 20 wt. % of the one or more cyclic carbonates, based on the total weight of the compositions. In further embodiments, the compositions include about 1 to about 18wt. %, about 1 to about 15 wt. %, about 1 to about 12 wt. %, about 2 to about 20 wt. %, about 2 to about 18 wt. %, about 2 to about 15 wt. %, about 2 to about 12 wt. %, about 5 to about 20 wt. %, about 5 to about 18 wt. %, about 5 to about 15 wt. %, about 5 to about 12 wt. %, about 8 to about 20 wt. %, about 8 to about 18 wt. %, about 8 to about 15 wt. %, or about 8 to about 12 wt. %, based on the total weight of the compositions.

### (b) Silicones

According to certain embodiments, the silicone is selected from dimethicones, dimethiconols, aminosilicones, and mixtures thereof. The dimethiconols are hydroxyl terminated dimethylsilicones and may be represented by the general chemical formulas

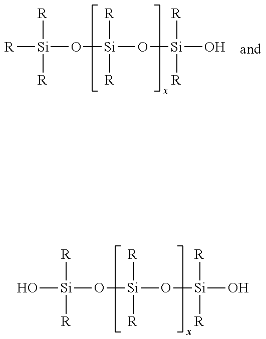

wherein R is an alkyl group (preferably R is methyl or ethyl, more preferably methyl) and x is an integer up to about 500, chosen to achieve the desired molecular weight. Dimethicones generally have similar structures, however lack the hydroxyl functional groups.

According to certain notable embodiments, the silicone is an aminosilicone. The term “amino-functionalized silicone” or “amino silicones” means a silicone containing at least one primary amino, secondary amino, tertiary amino and/or quaternary ammonium group. The structure of the amino-functionalized silicone may be linear or branched, cyclic or non-cyclic. The amino functional group may be at any position in the silicone molecule, preferably at the end of the backbone (for example, in the case of amodimethicones) and/or in the side chain.

In some instances, an amino-functionalized silicones is selected from compounds having the following formula:

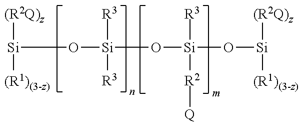

wherein each R¹ is independently selected from a C₁₋₃₀ alkyl group, a C₁₋₃₀ alkoxy group, a C₅₋₃₀ aryl group, a C₆₋₃₀ aralkyl group, a C₆₋₃₀ aralkyloxy group, a C₁₋₃₀ alkaryl group, a C₁₋₃₀ alkoxyaryl group, and a hydroxy group (preferably, each R¹ is independently selected from a C₁₋₃₀ alkyl group, a C₁₋₃₀ alkoxy group and a hydroxy group);
each R² is independently a divalent alkylene radical having one to ten carbon atoms (preferably, R² is a divalent alkylene radical having three to six carbon atoms);
each R³ is independently selected from a C₁₋₃₀ alkyl group, a C₅₋₃₀ aryl group, a C₆₋₃₀ aralkyl group and a C₁₋₃₀ alkaryl group (preferably, each R³ is independently selected from of a C₁₋₃₀ alkyl group);
Q is a monovalent radical selected from —NR⁴₂ and —NR⁴ (CH₂)ₓNR⁴₂;
each R⁴ is independently selected from a hydrogen and a C₁₋₄ alkyl group;
x is 2 to 6;
z is 0 or 1;
n is 25 to 3,000 (preferably, 25 to 2,000; more preferably, 25 to 1,000; most preferably 25 to 500); and
m is 0 to 3,000 (preferably, 0 to 2,000; more preferably, 0 to 1,000; most preferably, 0 to 100);
with the proviso that at least 50 mol % of the total number of R¹ and R³ groups are methyl and with the proviso that when m is 0, z is 1.

Preferred R¹ groups include methyl, methoxy, ethyl, ethoxy, propyl, propoxy, isopropyl, isopropoxy, butyl, butoxy, isobutyl, isobutoxy, phenyl, xenyl, benzyl, phenylethyl, tolyl and hydoxy. Preferred R² divalent alkylene radicals include trimethylene, tetramethylene, pentamethylene, —CH₂CH(CH₃)CH₂ and CH₂CH₂CH(CH₃)CH₂.

Preferred R³ groups include methyl, ethyl, propyl, isopropyl, butyl, isobutyl, phenyl, xenyl, benzyl, phenylethyl and tolyl. Preferred R⁴ groups include methyl, ethyl, propyl, isopropyl, butyl and isobutyl. When z is 0, the amino-functionalized silicine has only pendant amine functional substituents in the polymer chain. When z is 1, the amino-functional silicone may have only terminal amine functional substituents (e.g., m=0) or may have both terminal and pendant amine functional substituents in the polymer chain (e.g., m>0). Preferably, n+m is 50 to 1,000. More preferably, n+m is 50 to 750. Still more preferably, n+m is 50 to 500. Most preferably, n+m is 50 to 250.

In some instances, the amino-functionalized silicones are alkoxylated and/or hydroxylated amino silicones. Suitable alkoxylated and/or hydroxylated amino silicones may be selected from compounds of the following formula:

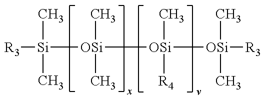

wherein R₃ is hydroxyl or OR₅, R₅ is a C₁ to C₄ alkyl group, R₄ is a group with structure according to the following formula:

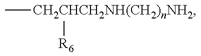

R₆ is a C₁ to C₄ alkyl, n is a 1 to 4, x is the same as “n” described above, and y is the same as “m” described above.

The silicone may be a polysiloxane corresponding to the following formula:

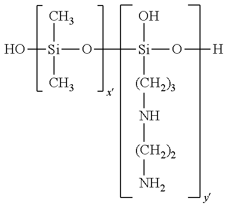

in which x′ and y′ are integers such that the weight-average molecular weight (Mw) is comprised between about 5000 and 500 000;
b) amino silicones corresponding to following formula:

R′ₐG₃₋ₐ-Si(OSiG₂)ₙ-(OSiG_{b}R′_{2-b})ₘ—O—SiG₃₋ₐ-R′ₐ
in which:
G, which may be identical or different, designate a hydrogen atom, or a phenyl, OH or C₁-C₈ alkyl group, for example methyl, or C₁-C₈ alkoxy, for example methoxy,
a, which may be identical or different, denote the number 0 or an integer from 1 to 3, in particular 0;
b denotes 0 or 1, and in particular 1;
m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10;
R′, which may be identical or different, denote a monovalent radical having formula —CqH₂qL in which q is a number ranging from 2 to 8 and L is an optionally quaternized amino group chosen from the following groups:

—NR″-Q-N(R″)₂

—N(R″)₂

—N+(R″)₃A-

—N+H(R″)₂ A-

—N+H₂(R″)A-

—N(R″)-Q-N+R″H₂A-

—NR″-Q-N+(R″)₂HA-

—NR″-Q-N+(R″)₃A-,
in which R″, which may be identical or different, denote hydrogen, phenyl, benzyl, or a saturated monovalent hydrocarbon-based radical, for example a C₁-C₂₀ alkyl radical; Q denotes a linear or branched CrH₂ᵣ group, r being an integer ranging from 2 to 6, preferably from 2 to 4; and A- represents a cosmetically acceptable ion, in particular a halide such as fluoride, chloride, bromide or iodide.

Another group of amino silicones corresponding to this definition is represented by silicones having the following formula:

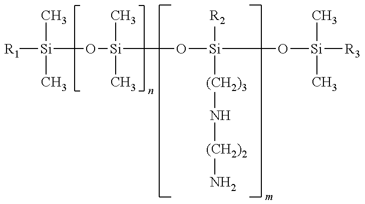

in which:
m and n are numbers such that the sum (n+m) can range from 1 to 1000, in particular from 50 to 250 and more particularly from 100 to 200, it being possible for n to denote a number from 0 to 999 and in particular from 49 to 249, and more particularly from 125 to 175, and for m to denote a number from 1 to 1000 and in particular from 1 to 10, and more particularly from 1 to 5;
R₁, R₂, R₃, which may be identical or different, represent a hydroxy or C₁-C₄ alkoxy radical, where at least one of the radicals R₁ to R₃ denotes an alkoxy radical.

The alkoxy radical is preferably a methoxy radical. The hydroxy/alkoxy mole ratio ranges preferably from 0.2:1 to 0.4:1 and preferably from 0.25:1 to 0.35:1 and more particularly equals 0.3:1. The weight-average molecular weight (Mw) of the silicone ranges preferably from 2,000 to 1,000,000, more particularly from 3,500 to 200,000.

Another group of amino silicones corresponding to this definition is represented by the following formula:

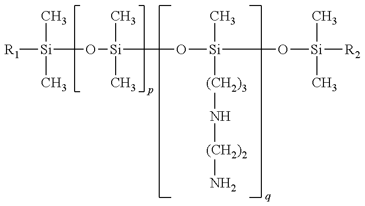

in which:
p and q are numbers such that the sum (p+q) ranges from 1 to 1000, particularly from 50 to 350, and more particularly from 150 to 250; it being possible for p to denote a number from 0 to 999 and in particular from 49 to 349, and more particularly from 159 to 239 and for q to denote a number from 1 to 1000, in particular from 1 to 10, and more particularly from 1 to 5;
R₁, R₂, which may be the same or different, represent a hydroxy or C₁-C₄ alkoxy radical, where at least one of the radicals R₁ or R₂ denotes an alkoxy radical.

The alkoxy radical is preferably a methoxy radical. The hydroxy/alkoxy mole ratio ranges generally from 1:0.8 to 1:1.1 and preferably from 1:0.9 to 1:1 and more particularly equals 1:0.95.

Another group of amino silicones is represented by the following formula:

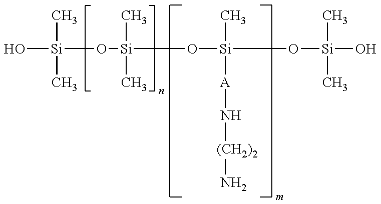

in which:
m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10;
A denotes a linear or branched alkylene radical containing from 4 to 8 carbon atoms and preferably 4 carbon atoms. This radical is preferably linear.

The weight-average molecular weight (Mw) of these amino silicones ranges preferably from 2000 to 1 000 000 and even more particularly from 3500 to 200 000.

Another group of amino silicones is represented by the following formula:

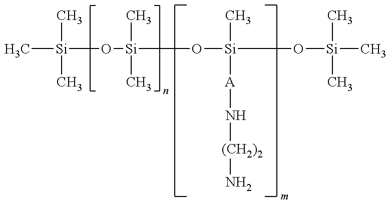

in which:
m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10;
A denotes a linear or branched alkylene radical containing from 4 to 8 carbon atoms and preferably 4 carbon atoms. This radical is preferably branched.

The weight-average molecular weight (Mw) of these amino silicones ranges preferably from 500 to 1 000 000 and even more particularly from 1000 to 200 000.

Another group of amino silicones is represented by the following formula:

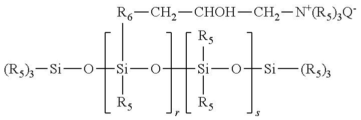

in which:
R₅ represents a monovalent hydrocarbon-based radical containing from 1 to 18 carbon atoms, and in particular a C₁-C₁₈ alkyl or C₂-C₁₈ alkenyl radical, for example methyl;
R₆ represents a divalent hydrocarbon-based radical, in particular a C₁-C₁₈ alkylene radical or a divalent C₁-C₁₈, for example C₁-C₈, alkylenoxy radical linked to the Si via an SiC bond;
Q- is an anion such as a halide ion, in particular chloride, or an organic acid salt (for example acetate);
r represents a mean statistical value from 2 to 20 and in particular from 2 to 8;
s represents a mean statistical value from 20 to 200 and in particular from 20 to 50.

Such amino silicones are described more particularly in patent U.S. Pat. No. 4,185,087.

A group of quaternary ammonium silicones is represented by the following formula:

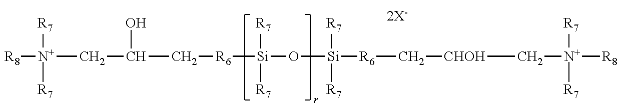

in which:
R₇, which may be identical or different, represent a monovalent hydrocarbon-based radical containing from 1 to 18 carbon atoms, and in particular a C₁-C₁₈ alkyl radical, a C₂-C₁₈ alkenyl radical or a ring containing 5 or 6 carbon atoms, for example methyl;
R₆ represents a divalent hydrocarbon-based radical, in particular a C₁-C₁₈ alkylene radical or a divalent C₁-C₁₈, for example C₁-C₈, alkylenoxy radical linked to the Si via an SiC bond;
R₈, which may be identical or different, represent a hydrogen atom, a monovalent hydrocarbon-based radical containing from 1 to 18 carbon atoms, and in particular a C₁-C₁₈ alkyl radical, a C₂-C₁₈ alkenyl radical or a —R₆—NHCOR₇ radical;
X-is an anion such as a halide ion, in particular chloride, or an organic acid salt (for example acetate);
r represents a mean statistical value from 2 to 200 and in particular from 5 to 100. These silicones are described, for example, in patent application EP-A 0530974.

A group of quaternary ammonium silicones is represented by the following formula:

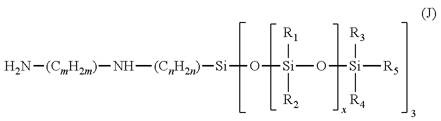

in which:
R₁, R₂, R₃ and R₄, which may be identical or different, denote a C₁-C₄ alkyl radical or a phenyl group;
R₅ denotes a C₁-C₄ alkyl radical or a hydroxyl group;
n is an integer ranging from 1 to 5;
m is an integer ranging from 1 to 5;
and in which x is chosen such that the amine number is between 0.01 and 1 meq/g;
multiblockpolyoxyalkylenated amino silicones, of type (AB)n, A being a polysiloxane block and B being a polyoxyalkylenated block containing at least one amine group.

Said silicones are preferably constituted of repeating units having the following general formulae:

[—(SiMe₂O)xSiMe₂-R—N(R″)—R′—O(C₂H₄O)ₐ(C₃H₆O)b-R′—N(H)—R—]

or alternatively

[—(SiMe₂O)xSiMe₂-R—N(R″)—R′—O(C₂H₄O)ₐ(C₃H₆O)b-]

in which:
a is an integer greater than or equal to 1, preferably ranging from 5 to 200, more particularly ranging from 10 to 100;
b is an integer comprised between 0 and 200, preferably ranging from 4 to 100, more particularly between from 5 and 30;
x is an integer ranging from 1 to 10 000, more particularly from 10 to 5000;
R″ is a hydrogen atom or a methyl;
R, which may be identical or different, represent a divalent linear or branched C₂-C₁₂ hydrocarbon-based radical, optionally including one or more heteroatoms such as oxygen; preferably, R denotes an ethylene radical, a linear or branched propylene radical, a linear or branched butylene radical, or a —CH₂CH₂CH₂OCH(OH)CH₂— radical; preferentially R denotes a —CH₂CH₂CH₂OCH(OH)CH₂— radical;
R′, which may be identical or different, represent a divalent linear or branched C₂-C₁₂ hydrocarbon-based radical, optionally including one or more heteroatoms such as oxygen; preferably, R′ denotes an ethylene radical, a linear or branched propylene radical, a linear or branched butylene radical, or a —CH₂CH₂CH₂OCH(OH)CH₂— radical; preferentially R′ denotes —CH(CH₃)—CH₂—.

The siloxane blocks preferably represent between 50 and 95 mol % of the total weight of the silicone, more particularly from 70 to 85 mol %.

The amine content is preferably between 0.02 and 0.5 meq/g of copolymer in a 30% solution in dipropylene glycol, more particularly between 0.05 and 0.2. The weight-average molecular weight (Mw) of the silicone oil is preferably comprised between 5000 and 1,000,000, more particularly between 10,000 and 200,000.

Non-limiting examples of amino-functionalized silicones include bis-hydroxy/methoxy amodimethicones, bis-cetearyl amodimethicone, amodimethicone, bis(C13-15 alkoxy) PG amodimethicones, aminopropyl phenyl trimethicones, aminopropyl dimethicones, bis-amino PEG/PPG-41/3 aminoethyl PG-propyl dimethicones, caprylyl methicones, and a mixture thereof. In some instances, a particularly useful amino-functionalized silicone is bis-hydroxy/methoxy amodimethicone, wherein X is isobutyl and one of the R is OH and the other is OCH₃ in the above structure, also known as “Bis-Hydroxy/Methoxy Amodimethicone” and “3-[(2-aminoethyl)amino]-2-methylpropyl Me, di-Me, [(hydroxydimethylsilyl)oxy]- and [(methoxydimethylsilyl)oxy]-terminated.” Bis-hydroxy/methoxy amodimethicone is commercially available under the tradename DOWSIL AP-8087 FLUID from The Dow Chemical Company. A particularly preferred amino-functionalized silicone is amodimethicone” A non-limiting example of amodimethicone products containing amino silicones having structure (D) re sold by Wacker under the name BELSIL ADM 652, BELSIL ADM 4000 E, or BELSIL ADM LOG 1. A product containing amino silicones having structure (E) is sold by Wacker under the name FLUID WR 1300. Additionally or alternative, the weight-average molecular weight (Mw) of the silicone ranges preferably from 2,000 to 200,000, even more particularly 5,000 to 100,000 and more particularly from 10,000 to 50,000.

In a preferred embodiment, the one or more amino-functionalized silicones are selected from amodimethicone, bis-hydroxy/methoxy amodimethicone, bis-cetearyl amodimethicone, bis(C13-15 alkoxy) PG amodimethicone, aminopropyl phenyl trimethicone, aminopropyl dimethicone, bis-amino PEG/PPG-41/3 aminoethyl PG-propyl dimethicone, or a mixture thereof. In a further preferred embodiments, the amino-functionalized silicone is amodimethicone.

The total amount of the one or more amino-functionalized silicones in the leave-on finishing compositions will vary. Nonetheless, in various embodiments, the compositions include about 1 to about 10 wt. % of the one or more amino-functionalized silicones, based on the total weight of the leave-on finishing composition. In further embodiments, the leave-on finishing compositions include about 1 to about 8 wt. %, about 1 to about 6 wt. %, about 1 to about 5 wt. %, about 2 to about 10 wt. %, about 2 to about 8 wt. %, about 2 to about 6 wt. %, about 2 to about 5 wt. %, about 3 to about 10 wt. %, about 3 to about 8 wt. %, about 3 to about 6 wt. %, or about 3 to about 5 wt. %, based on the total weight of the leave-on finishing composition.

### Ratio of (a) to (b)

The weight ratio of the one or more cyclic carbonates (a) to the one or more amino-functionalized silicones (b) in the leave-on finishing compositions will vary. Nonetheless, in various embodiments, the weight ratio of the one or more cyclic carbonates (a) to the one or more amino-functionalized silicones (b) is about 20:1 to about 1:1 ((a): (b)). In further embodiments, the weight ratio of the one or more cyclic carbonates (a) to the one or more amino-functionalized silicones (b) is about 18:1 to about 1:1, about 15:1 to about 1:1, about 12:1 to about 1:1, about 10:1 to about 1:1, about 8:1 to about 1:1, about 6:1 to about 1:1, about 20:1 to about 2:1, about 18:1 to about 2:1, about 15:1 to about 2:1, about 12:1 to about 2:1, about 10:1 to about 2:1, about 8:1 to about 2:1, about 6:1 to about 2:1, about 20:1 to about 3:1, about 18:1 to about 3:1, about 15:1 to about 3:1, about 12:1 to about 3:1, about 10:1 to about 3:1, about 8:1 to about 3:1, about 6:1 to about 3:1, or about 5:1 to about 3:1.

### (c) Water

The total amount of water in the leave-on finishing compositions will vary. Nonetheless, in various embodiments, the compositions include about 50 to about 90 wt. % of water, based on the total weight of the compositions. In further embodiments, the compositions include about 60 to about 90 wt. %, about 70 to about 90 wt. %, about 50 to about 85 wt. %, about 60 to about 85 wt. %, about 70 to about 85 wt. %, about 50 to about 80 wt. %, about 60 to about 80 wt. %, about 70 to about 80 wt. %, about 65 to about 85 wt. %, based on the total weight of the compositions.

### (d) Polysaccharides

The term “polysaccharides” refers to compounds containing a backbone of repeating sugar (i.e., carbohydrate) units. The polysaccharides may be cationic, nonionic, or anionic. In various embodiments, the polysaccharides are preferably nonionic polysaccharides.

Nonlimiting examples of polysaccharides include starches, gums, celluloses, and a mixtures thereof. Nonlimiting examples of starches include modified starches, starch-based polymers, methylhydroxypropyl starch, potato starch, wheat starch, rice starch, starch crosslinked with octenyl succinic anhydride (sold under the name Dry-Flo by National Starch), starch oxide, dialdehyde starch, dextrin, British gum, acetyl starch, starch phosphate, carboxymethyl starch, hydroxyethyl starch, and hydroxypropyl starch.

Nonlimiting examples of cellulose-based polymers include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Also useful herein are the alkyl-substituted celluloses. Preferred among the alkyl hydroxyalkyl cellulose ethers is the material given the CTFA designation cetyl hydroxyethylcellulose, which is the ether of cetyl alcohol and hydroxyethylcellulose. This material is sold under the tradename NATROSOL CS Plus from Aqualon Corporation.

In various embodiments, the polysaccharides are preferably selected from scleroglucans comprising a linear chain of (1-3) linked glucose units with a (1-6) linked glucose every three units, a commercially available example of which is CLEAROGEL. CS1 1 from Michel Mercier Products Inc.

Nonlimiting examples of gums include acacia, agar, algin, alginic acid, ammonium alginate, amylopectin, calcium alginate, calcium carrageenan, carnitine, carrageenan, dextrin, gelatin, gellan gum, guar gum, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, biosacharide gum, and mixtures thereof.

In various embodiments, the one or more polysaccharides are selected from hydroxypropyl starch phosphate, potato starch (modified or unmodified), wheat starch, rice starch, hydroxyethyl cellulose, guar gum, hydroxypropyl guar, xanthan gum, sclerotium gum, and a mixture thereof. In yet further embodiments, the polysaccharide is hydroxypropyl starch phosphate. Hydroxypropyl starch phosphate is sold under the tradename of STRUCTURE ZEA by the company Akzo Nobel. In a preferred embodiment, at least one of the one or more polysaccharides is sclerotium gum.

The total amount of the one or more polysaccharides in the compositions will vary. Nonetheless, in various embodiments, the compositions include about 0.1 to about 8 wt. % of the one or more polysaccharides, based on the total weight of the composition. In further embodiments, the compositions include about 0.1 to about 6 wt. %, about 0.1 to about 5 wt. %, about 0.1 to about 4 wt. %, about 0.1 to about 3 wt. %, about 0.1 to about 2 wt. %, about 0.2 to about 8 wt. %, about 0.2 to about 6 wt. %, about 0.2 to about 5 wt. %, about 0.2 to about 4 wt. %, about 0.2 to about 3 wt. %, about 0.2 to about 2 wt. %, about 0.3 to about 8 wt. %, about 0.3 to about 6 wt. %, about 0.3 to about 5 wt. %, about 0.3 to about 5 wt. %, about 0.3 to about 4 wt. %, about 0.3 to about 3 wt. %, or about 0.3 to about 2 wt. %, based on the total weight of the composition.

### (e) Nonionic Associative Polymeric Thickeners

The leave-on finishing compositions may further include an associative thickener—i.e., one capable of reversibly associating with itself or with other molecules or particles. This physical association provides thickening and can give rise to thixotropic or shear-thinning macromolecular systems, i.e. systems whose viscosity depends on the shear forces to which they are subjected.

In various embodiments, the nonionic associative polymer thickener is a nonionic associative polyurethane thickener, preferably a nonionic associative polyurethane/polyether. The nonionic polyurethane/polyethers may have both at least one hydrophilic moiety and at least one hydrophobic moiety. More particularly, said polymers may contain in their chain both hydrophilic sequences most often of a polyoxyethylenated nature and hydrophobic sequences which may be aliphatic linkages alone and/or cycloaliphatic and/or aromatic linkages. In various embodiments, these polyether-polyurethanes comprise at least two lipophilic hydrocarbon chains, having from 6 to 30 carbon atoms, preferably from 6 to 20, separated by a hydrophilic sequence, it being possible for the hydrocarbon chains to be pendent chains or chains at the end of a hydrophilic sequence. In particular, it is possible for one or more pendent chains to be envisaged. In addition, the polymer may comprise a hydrocarbon chain at one end or at both ends of a hydrophilic sequence.

The polyether-polyurethanes may be polyblocks, in particular in triblock form. The hydrophobic sequences may be at each end of the chain (for example: triblock copolymer with hydrophilic central sequence) or distributed both at the ends and in the chain (polyblock copolymers for example). These same polymers may also be in the form of graft units or may be star shaped.

The nonionic polyether/polyurethanes containing a fatty chain may be triblock copolymers whose hydrophilic sequence is a polyoxyethylenated chain comprising from 50 to 1000 oxyethylenated groups. The nonionic polyether-polyurethanes comprise a urethane bond between the hydrophilic sequences, hence the origin of the name. By extension, those whose hydrophilic sequences are linked by other chemical bonds to the hydrophobic sequences are also included among the nonionic polyether-polyurethanes containing a hydrophobic chain.

Nonlimiting examples of nonionic polyether/polyurethanes containing a hydrophobic chain include Rheolate® 205 containing a urea functional group sold by the company RHEOX or else the Rheolates® 208, 204 or 212, as well as Acrysol RM 184®. Additional products include ELFACOS T210® containing a C12-C14 alkyl chain and the product ELFACOS T212® containing a C18 alkyl chain from AKZO. The product DW 1206B® from ROHM & HAAS containing a C20 alkyl chain and with a urethane bond, sold at 20% dry matter content in water, may also be used.

It is also possible to use solutions or dispersions of these polymers in particular in water or in an aqueous-alcoholic medium. By way of examples of such polymers, there may be mentioned Rheolate® 255, Rheolate® 278 and Rheolate® 244 sold by the company RHEOX. It is also possible to use the product DW 1206F and DW 1206J provided by the company ROHM & HAAS.

As the above-described polyether/polyurethanes, mention may be made of polyurethane/polyethers comprising in their chain at least one polyoxyethylenated hydrophilic block and at least one of hydrophobic blocks containing at least one sequence chosen from aliphatic sequences, cycloaliphatic sequences, and aromatic sequences. In various embodiments, it is preferable that the polyurethane/polyethers comprise at least two hydrocarbon-based lipophilic chains having from 8 to 30 carbon atoms, separated by a hydrophilic block, and wherein the hydrocarbon-based chains are chosen from pendent chains and chains at the end of the hydrophilic block.

In a preferred embodiment, use is made a polyurethane/polyether that may be obtained by polycondensation of at least three compounds comprising (i) at least one polyethylene glycol comprising from 150 to 180 mol of ethylene oxide, (ii) a polyoxyethylenated stearyl alcohol comprising 100 mol of ethylene oxide, and (iii) a diisocyanate. Such polyurethane/polyethers are sold especially by the company Element is under the name Rheolate FX 1100® and Rheoluxe 811®, which is a polycondensate of polyethylene glycol containing 136 mol of ethylene oxide, of stearyl alcohol polyoxyethylenated with 100 mol of ethylene oxide and of hexamethylene diisocyanate (HDI) with a weight-average molecular weight of 40000 (INCI name: PEG-136/Steareth-100/HDI Copolymer).

According to another embodiment, use will be made of a polyurethane/polyether that may be obtained by polycondensation of at least three compounds comprising (i) at least one polyethylene glycol comprising from 150 to 180 mol of ethylene oxide, (ii) stearyl alcohol or decyl alcohol, and (iii) at least one diisocyanate. Such polyurethane/polyethers are sold in particular by the company Rohm & Haas under the names Aculyn 46® and Aculyn 44®.

Aculyn 46® having the INCI name: PEG-150/Stearyl Alcohol/SMDI Copolymer, is a polycondensate of polyethylene glycol comprising 150 or 180 mol of ethylene oxide, of stearyl alcohol and of methylenebis(4-cyclohexyl isocyanate) (SMDI) at 15% by weight in a matrix of maltodextrin (4%) and water (81%) (INCI name: PEG-150/Stearyl Alcohol/SMDI Copolymer). Aculyn 44® (PEG-150/Decyl Alcohol/SMDI Copolymer) is a polycondensate of polyethylene glycol comprising 150 or 180 mol of ethylene oxide, of decyl alcohol and of methylenebis(4-cyclohexyl isocyanate) (SMDI) at 35% by weight in a mixture of propylene glycol (39%) and water (26%) (INCI name: PEG-150/Decyl Alcohol/SMDI Copolymer).

As the associative polyurethanes, it may be preferable to use a compound represented by the following formula (1):

R¹—{(O—R²)_{K}—OCONH—R³[—NHCOO—(R⁴—O)ₙ—R⁵]ₕ}ₘ (1)
wherein R¹ represents a hydrocarbon group, R² and R⁴ independently represent alkylene groups having 2 to 4 carbon atoms, which alkylene groups may be identical or different from each other, or a phenylethylene group, R³ represents a hydrocarbon group, which may optionally have a urethane bond, R5 represents a branched chain or secondary hydrocarbon group, “m” represents a number of at least 2, “h” represents a number of at least 1, “k” represents a number within the range of 1 to 500, and “n” represents a number within the range of 1 to 200.

The hydrophobically modified polyurethane represented by the general formula (1) shown above is obtained by, for example, reacting at least one polyether polyol that is represented by the formula R¹—[(O—R²)ₖ—OH]ₘ, at least one polyisocyanate that is represented by the formula R³—(NCO)ₕ₊₁, and at least one polymonoalcohol that is represented by the formula HO—(R⁴—O)ₙ—R⁵. In such cases, R¹ to R⁵ in the general formula (1) are determined by the compounds R¹—[(O—R²)ₖ—OH]ₘ, R³—(NCO)ₕ₊₁ and HO—(R⁴—O)ₙ—R⁵. The loading ratios among the three compounds are not limited particularly and should preferably be such that the ratio of the isocyanate group derived from the polyisocyanate to the hydroxyl group derived from the polyether polyol and the polyether monoalcohol is selected within the range of NCO/OH of between 0.8:1 and 1.4:1.

The polyether polyol compound that is represented by the formula R¹—[(O—R²)ₖ—OH]ₘ and that may be used preferably for obtaining the associative thickener represented by the general formula (1) may be obtained from addition polymerization of an m-hydric polyol with an alkylene oxide, such as ethylene oxide, propylene oxide, butylene oxide, or epichlorohydrin, or with styrene oxide, and the like.

The polyols should preferably be di- to octa-hydric polyols. Examples of the di- to octa-hydric polyols include dihydric alcohols, such as ethylene glycol, propylene glycol, butylene glycol, hexamethylene glycol, and neopenthyl glycol; trihydric alcohols, such as glycerol, trioxy isobutane, 1,2,3-butanetriol, 1,2,3-pentanetriol, 2-methyl-1,2,3-propanetriol, 2-methyl-2,3,4-butanetriol, 2-ethyl-1,2,3-butanetriol, 2,3,4-pentanetriol, 2,3,4-hexanetriol, 4-propyl-3,4,5-heptanetriol, 2,4-dimethyl-2,3,4-pentanetriol, pentamethylglycerol, pentaglycerol, 1,2,4-butanetriol, 1,2,4-pentanetriol, trimethylolethane, and trimethylolpropane; tetrahydric alcohols, such as pentaerythritol, 1,2,3,4-pentanetetrol, 2,3,4,5-hexanetetrol, 1,2,4,5-pentanetetrol, and 1,3,4,5-hexanetetrol; pentahydric alcohols, such as adonitol, arabitol, and xylitol; hexahydric alcohols, such as dipentaerythritol, sorbitol, mannitol, and iditol; and octahydric alcohols, such as sucrose.

Also, R² is determined by the alkylene oxide, styrene oxide, or the like, which is subjected to the addition. Particularly, for availability and excellent effects, an alkylene oxide having 2 to 4 carbon atoms, or styrene oxide is preferable.

The alkylene oxide, styrene oxide, or the like, to be subjected to the addition may be subjected to single polymerization, or random polymerization or block polymerization of at least two members. The procedure for the addition may be a conventional procedure. Also, the polymerization degree K may be selected within the range of 0 to 1,000, preferably within the range of 1 to 500, and more preferably within the range of 10 to 200. Further, the ratio of the ethylene group occupying R2 should preferably be within the range of 50 to 100 mass % with respect to the total quantity of R2. In such cases, the associative thickener appropriate for the purposes of the present invention is obtained.

Furthermore, the molecular weight of the polyether polyol compound that is represented by the formula R¹—[(O—R²)ₖ—OH]ₘ, should preferably be selected within the range of 500 to 100,000, and should more preferably be selected within the range of 1,000 to 50,000.

The polyisocyanate that is represented by the formula R³—(NCO)ₕ₊₁ and that may be used preferably for obtaining the hydrophobically modified polyether urethane represented by the general formula (1) employed in accordance with the present invention is not limited particularly in so far as the polyisocyanate has at least two isocyanate groups in the molecule. Examples of the polyisocyanates include aliphatic diisocyanates, aromatic diisocyanates, alicyclic diisocyanates, biphenyl diisocyanate, phenylmethane diisocyanate, phenylmethane triisocyanate, and phenylmethane tetraisocyanate.

Also, it is possible to employ dimers and trimers (isocyanurate bonds) of the above-enumerated polyisocyanates. Further, it is possible to employ biuret obtained by a reaction with an amine.

Furthermore, it is possible to employ a polyisocyanate having a urethane bond obtained by a reaction of the aforesaid polyisocyanate compound and a polyol. As the polyol, di- to octa-hydric polyols are preferable, and the above-enumerated polyols are preferable. In cases where a tri- or higher-hydric polyisocyanate is used as the polyisocyanate that is represented by the formula R3-(NCO)n+1, it is preferable to employ the aforesaid polyisocyanate having the urethane bond.

The polyether monoalcohol that is represented by the formula HO—(R⁴—O)ₙ—R⁵ and that may be used preferably for obtaining the hydrophobically modified polyether urethane represented by the general formula (1) employed in accordance with the present invention is not limited particularly in so far as the polyether monoalcohol is a polyether of a straight chain, branched chain, or secondary monohydric alcohol. The polyether monoalcohol may be obtained by addition polymerization of the straight chain, branched chain, or secondary monohydric alcohol with an alkylene oxide, such as ethylene oxide, propylene oxide, butylene oxide, or epichlorohydrin, or with styrene oxide, and the like.

The compound represented by the general formula (1) may be produced by, for example, heating at a temperature of 80 to 90° C. for 1 to 3 hours and thereby causing a reaction to occur in the same manner as that in the ordinary reaction of a polyether and an isocyanate.

As the compound represented by the general formula (1), polyethyleneglycol-240/decyltetradeceth-20/hexamethylene diisocyanate copolymer is preferable. The polyethyleneglycol-240/decyltetradeceth-20/hexamethylene diisocyanate copolymer is referred to also as PEG-240/HDI copolymer bis-decyltetradeceth-20 ether.

In various embodiments, it is preferable that the nonionic associative polyurethane thickener be selected from steareth-100/PEG-136/HDI copolymer sold by the company Rheox under the name of Rheolate FX 1100, PEG-240/HDI copolymer bis-decyltetradeceth-20 ether sold by the company Asahi Denka under the name of Adekanol GT-700, and mixtures thereof.

The total amount of the optional one or more nonionic associative polymeric thickeners will vary. Nonetheless, in various embodiments, the compositions include about 0.1 to about 8 wt. %, based on the total weight of the compositions. In further embodiments, the compositions include about 0.1 to about 5 wt. %, about 0.1 to about 4 wt. %, about 0.1 to about 3 wt. %, about 0.1 to about 2 wt. %, about 0.5 to about 8 wt. %, about 0.5 to about 5 wt. %, about 0.5 to about 4 wt. %, about 0.5 to about 3 wt. %, about 0.5 to about 2 wt. %, based on the total weight of the compositions.

### (f) Nonionic Surfactants or Emulsifiers

The leave-on finishing compositions may further include a surfactant or emulsifier such as a nonionic surfactant or emulsifier. The terms “nonionic surfactant” and “nonionic emulsifier” are used interchangeably in the instant disclosure and therefore can be referred to as “nonionic emulsifying surfactants.” The nonionic surfactant or emulsifier may have an HLB (hydrophilic-lipophilic balance) ranging from 1 to 7.9 or greater than or equal to 8. “HLB” refers to the “hydrophilic-lipophilic balance” associated with nonionic surfactants or emulsifiers. In particular, “HLB” value relates to the ratio of hydrophilic groups and lipophilic groups in emulsifiers, and also relates to solubility of the emulsifiers. Lower HLB emulsifiers (such as those with HLB values ranging from 1 to 7.9) are more soluble in oils (lipophilic material) and are more appropriate for use in water-in-oil (W/O) emulsions. Higher HLB emulsifiers (such as those with HLB values higher than 8) are more soluble in water (hydrophilic material) and are more appropriate for oil-in-water (O/W) emulsions.

Nonlimiting examples of nonionic surfactants or emulsifiers include alkyl and polyalkyl esters of poly(ethylene oxide), alkyl and polyalkyl ethers of poly(ethylene oxide), optionally polyoxyethylenated alkyl and polyalkyl esters of sorbitan, optionally polyoxyethylenated alkyl and polyalkyl ethers of sorbitan, alkyl and polyalkyl glycosides or polyglycosides, in particular alkyl and polyalkyl glucosides or polyglucosides, alkyl and polyalkyl esters of sucrose, optionally polyoxyethylenated alkyl and polyalkyl esters of glycerol, and optionally polyoxyethylenated alkyl and polyalkyl ethers of glycerol, and mixtures thereof. Preferably, the non-ionic surfactant(s) may be chosen from alkyl and polyalkyl esters of poly(ethylene oxide), alkyl and polyalkyl ethers of poly(ethylene oxide), optionally polyoxyethylenated alkyl and polyalkyl esters of sorbitan, optionally polyoxyethylenated alkyl and polyalkyl ethers of sorbitan, optionally polyoxyethylenated alkyl and polyalkyl esters of glycerol, and optionally polyoxyethylenated alkyl and polyalkyl ethers of glycerol, and mixtures thereof.
(1) Alkyl and polyalkyl esters of poly(ethylene oxide) that are preferably used are those containing at least one C8-C30 alkyl radical, with a number of ethylene oxide (EO) units ranging from 2 to 200. Mention may be made, for example, of (INCI name) PEG-20 stearate, PEG-40 stearate, PEG-100 stearate, PEG-20 laurate, PEG-8 laurate, PEG-40 laurate, PEG-150 distearate, PEG-7 cocoate, PEG-9 cococate, PEG-8 oleate, PEG-10 oleate and PEG-40 hydrogenated castor oil.
(2) Alkyl and polyalkyl ethers of poly(ethylene oxide) that are preferably used are those containing at least one C8-C30 alkyl radical, with a number of ethylene oxide (EO) units ranging from 3 to 200. Mention may be made, for example, of laureth-3, laureth-4, laureth-7, laureth-23, ceteth-5, ceteth-7, ceteth-15, ceteth-23, oleth-5, oleth-7, oleth-10, oleth-12, oleth-20, oleth-50, phytosterol 30 EO, steareth-6, steareth-20, steareth-21, steareth-40, steareth-100, beheneth 100, ceteareth-7, ceteareth-10, ceteareth-15, ceteareth-25, pareth-3, pareth-23, C12-15 pareth-3, C12-13 pareth-4, C12-13 pareth-23, trideceth-3, trideceth-4, trideceth-5, trideceth-6, trideceth-7 and trideceth-10, and mixtures thereof.
(3) Polyoxyethylenated alkyl and polyalkyl esters of sorbitan that are preferably used are those with a number of ethylene oxide (EO) units ranging from 0 to 100. Mention may be made, for example, of sorbitan laurate, sorbitan laurate 4 EO, sorbitan laurate 20 EO (polysorbate 20), sorbitan palmitate 20 EO (polysorbate 40), sorbitan stearate 20 EO (polysorbate 60), sorbitan oleate 20 EO (polysorbate 80) and sorbitan trioleate 20 EO (polysorbate 85).
(4) Polyoxyethylenated alkyl and polyalkyl ethers of sorbitan that are preferably used are those with a number of ethylene oxide (EO) units ranging from 0 to 100.

The compositions of the instant disclosure may include one or more alkanolamides. Non-limiting examples alkanolamides include fatty acid alkanolamides. The fatty acid alkanolamides may be fatty acid monoalkanolamides or fatty acid dialkanolamides or fatty acid isoalkanolamides, and may have a C₂₋₈ hydroxyalkyl group (the C₂₋₈ chain can be substituted with one or more than one —OH group). Non-limiting examples include fatty acid diethanolamides (DEA) or fatty acid monoethanolamides (MEA), fatty acid monoisopropanolamides (MIPA), fatty acid diisopropanolamides (DIPA), and fatty acid glucamides (acyl glucamides).

Suitable fatty acid alkanolamides include those formed by reacting an alkanolamine and a C6-C36 fatty acid. Examples include, but are not limited to: oleic acid diethanolamide, myristic acid monoethanolamide, soya fatty acids diethanolamide, stearic acid ethanolamide, oleic acid monoisopropanolamide, linoleic acid diethanolamide, stearic acid monoethanolamide (Stearamide MEA), behenic acid monoethanolamide, isostearic acid monoisopropanolamide (isostearamide MIPA), erucic acid diethanolamide, ricinoleic acid monoethanolamide, coconut fatty acid monoisopropanolamide (cocoamide MIPA), coconut acid monoethanolamide (Cocamide MEA), palm kernel fatty acid diethanolamide, coconut fatty acid diethanolamide, lauric diethanolamide, polyoxyethylene coconut fatty acid monoethanolamide, coconut fatty acid monoethanolamide, lauric monoethanolamide, lauric acid monoisopropanolamide (lauramide MIPA), myristic acid monoisopropanolamide (Myristamide MIPA), coconut fatty acid diisopropanolamide (cocamide DIPA), and mixtures thereof.

In some instances, the fatty acid alkanolamides preferably include cocamide MIPA, cocamide DEA, cocamide MEA, cocamide DIPA, and mixtures thereof. In particular, the fatty acid alkanolamide may be cocamide MIPA, which is commercially available under the tradename EMPILAN from Innospec Active Chemicals.

Fatty acid alkanolamides include those of the following structure:

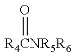

wherein R₄ is an alkyl chain of 4 to 20 carbon atoms (R₄ may be, for example, selected from lauric acid, coconut acid, palmitic acid, myristic acid, behenic acid, babassu fatty acid, isostearic acid, stearic acid, corn fatty acid, soy fatty acid, shea butter fatty acids, caprylic acid, capric acid, and mixtures thereof);
R₆ is selected from —CH₂OH, —CH₂CH₂OH, —CH₂CH₂CH₂OH, —CH₂(CHOH)₄CH₂OH, -benzyl, and mixtures thereof;
R₆ is selected from —H, —CH₃, —CH₂OH, —CH₂CH₃, —CH₂CH₂OH, —CH₂CH₂CH₂OH, —CH₂(CHOH)₄CH₂OH, -benzyl, and mixtures thereof.

In some instances, the one or more of the fatty acid alkanolamides include one or more acyl glucamides, for example, acyl glucamides having a carbon chain length of 8 to 20. Non-limiting examples include lauroyl/myristoyl methyl glucamide, capryloyl/capryl methyl glucamide, lauroyl methyl glucamide, myristoyl methyl glucamide, capryloyl methyl glucamide, capryl methyl glucamide, cocoyl methyl glucamide, capryloyl/caproyl methyl glucamide, cocoyl methyl glucamide, lauryl methylglucamide, oleoyl methylglucamide oleate, stearoyl methylglucamide stearate, sunfloweroyl methylglucamide, and tocopheryl succinate methylglucamide.

The compositions of the instant disclosure may include one or more alkyl polyglucosides. Non-limiting examples of alkyl polyglucosides include those having the following formula:

R¹—O—(R²O)ₙ—Z(x)
wherein R¹ is an alkyl group having 8-18 carbon atoms;
R² is an ethylene or propylene group;
Z is a saccharide group with 5 to 6 carbon atoms;
n is an integer from 0 to 10; and
x is an integer from 1 to 5.

Useful alkyl poly glucosides include lauryl glucoside, octyl glucoside, decyl glucoside, coco glucoside, caprylyl/capryl glucoside, and sodium lauryl glucose carboxylate. Typically, the at least one alkyl poly glucoside compound is selected from the group consisting of lauryl glucoside, decyl glucoside and coco glucoside. In some instances, decyl glucoside is particularly preferred.

The compositions of the instant disclosure may include one or more miscellaneous nonionic surfactants or emulsifiers. Nonlimiting examples include alcohols, alpha-diols, alkylphenols and esters of fatty acids, being ethoxylated, propoxylated or glycerolated and having at least one fatty chain comprising, for example, from 8 to 18 carbon atoms, it being possible for the number of ethylene oxide or propylene oxide groups to range from 2 to 50, and for the number of glycerol groups to range from 1 to 30. Maltose derivatives may also be mentioned. Non-limiting mention may also be made of copolymers of ethylene oxide and/or of propylene oxide; condensates of ethylene oxide and/or of propylene oxide with fatty alcohols; polyethoxylated fatty amides comprising, for example, from 2 to 30 mol of ethylene oxide; polyglycerolated fatty amides comprising, for example, from 1.5 to 5 glycerol groups, such as from 1.5 to 4; ethoxylated fatty acid esters of sorbitan comprising from 2 to 30 mol of ethylene oxide; ethoxylated oils from plant origin; fatty acid esters of sucrose; fatty acid esters of polyethylene glycol; polyethoxylated fatty acid mono or diesters of glycerol (C₆-C₂₄)alkylpolyglycosides; N—(C₆-C₂₄)alkylglucamine derivatives, amine oxides such as (C₁₀-C₁₄)alkylamine oxides or N—(C₁₀-C₁₄) acylaminopropylmorpholine oxides; and mixtures thereof.

Such nonionic surfactants may preferably be chosen from polyoxyalkylenated or polyglycerolated nonionic surfactants. The oxyalkylene units are more particularly oxyethylene or oxypropylene units, or a combination thereof, and are preferably oxyethylene units.

In some cases, the nonionic surfactant may be selected from esters of polyols with fatty acids with a saturated or unsaturated chain containing for example from 8 to 24 carbon atoms, preferably 12 to 22 carbon atoms, and alkoxylated derivatives thereof, preferably with a number of alkyleneoxide of from 10 to 200, and more preferably from 10 to 100, such as glyceryl esters of a C₈-C₂₄, preferably C₁₂-C₂₂, fatty acid or acids and alkoxylated derivatives thereof, preferably with a number of alkyleneoxide of from 10 to 200, and more preferably from 10 to 100; polyethylene glycol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sorbitol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sugar (sucrose, glucose, alkylglycose) esters of a C₈-C₂₄, preferably C₁₂-C₂₂, fatty acid or acids and alkoxylated derivatives thereof, preferably with a number of alkyleneoxide of from 10 to 200, and more preferably from 10 to 100; ethers of fatty alcohols; ethers of sugar and a C₈-C₂₄, preferably C₁₂-C₂₂, fatty alcohol or alcohols; and mixtures thereof.

Examples of ethoxylated fatty esters that may be mentioned include the adducts of ethylene oxide with esters of lauric acid, palmitic acid, stearic acid or behenic acid, and mixtures thereof, especially those containing from 9 to 100 oxyethylene groups, such as PEG-9 to PEG-50 laurate (as the CTFA names: PEG-9 laurate to PEG-50 laurate); PEG-9 to PEG-50 palmitate (as the CTFA names: PEG-9 palmitate to PEG-50 palmitate); PEG-9 to PEG-50 stearate (as the CTFA names: PEG-9 stearate to PEG-50 stearate); PEG-9 to PEG-50 palmitostearate; PEG-9 to PEG-50 behenate (as the CTFA names: PEG-9 behenate to PEG-50 behenate); polyethylene glycol 100 EO monostearate (CTFA name: PEG-100 stearate); and mixtures thereof.

As glyceryl esters of fatty acids, glyceryl stearate (glyceryl mono-, di- and/or tristearate) (CTFA name: glyceryl stearate) or glyceryl ricinoleate and mixtures thereof can in particular be cited.

As glyceryl esters of C₈-C₂₄ alkoxylated fatty acids, polyethoxylated glyceryl stearate (glyceryl mono-, di- and/or tristearate) such as PEG-20 glyceryl stearate can for example be cited.

Mixtures of these surfactants, such as for example the product containing glyceryl stearate and PEG-100 stearate, marketed under the name ARLACEL 165 by Uniqema, and the product containing glyceryl stearate (glyceryl mono- and distearate) and potassium stearate marketed under the name TEG1N by Goldschmidt (CTFA name: glyceryl stearate SE), can also be used.

The total amount of the one or more nonionic surfactants or emulsifiers in the compositions will vary. Nonetheless, in various embodiments, the compositions include about 0.1 to about 10 wt. % of the one or more nonionic surfactants or emulsifiers. In further embodiments, the compositions include about 0.1 to about 8 wt. %, about 0.1 to about 5 wt. %, about 0.1 to about 3 wt. %, about 0.2 to 10 wt. %, about 0.5 to about 10 wt. %, about 0.5 to about 8 wt. %, about 0.5 to about 5 wt. %, about 0.5 to about 3 wt. %, about 1 to about 10 wt. %, about 1 to about 8 wt. %, about 1 to about 5 wt. %, or about 1 to about 3 wt. %, based on the total weight of the compositions.

### (g) Non-Silicone-Based Fatty Compounds

The leave-on finishing compositions may further include a non-silicone-based fatty compound. The term “fatty compound” means an organic compound without silicone that is insoluble in water at ordinary temperature (25° C.) and at atmospheric pressure (760 mmHg), i.e. which has a solubility of less than 5%, preferably less than 1% and even more preferentially less than 0.1%. They have in their structure a hydrocarbon-based chain containing at least 6 carbon atoms.

More particularly, the one or more non-silicone-based fatty compounds may be selected from C6-C16 hydrocarbons, hydrocarbons containing more than 16 carbon atoms, non-silicone oils of animal origin, plant oils of triglyceride type, synthetic triglycerides, fluoro oils, fatty alcohols, non-salified fatty acids, fatty acid and/or fatty alcohol esters other than triglycerides and plant waxes, non-silicone waxes and silicones, and mixtures thereof.

Fatty alcohols, fatty esters and fatty acids more particularly contain one or more linear or branched, saturated or unsaturated hydrocarbon-based groups comprising 6 to 30 carbon atoms, which are optionally substituted, in particular, with one or more (in particular 1 to 4) hydroxyl groups. If they are unsaturated, these compounds may comprise one to three conjugated or unconjugated carbon-carbon double bonds.

As regards the C6-C16 hydrocarbons, they are linear, branched or optionally cyclic, and are preferably alkanes. Examples that may be mentioned include hexane, dodecane and isoparaffins such as isohexadecane and isodecane.

A hydrocarbon-based oil of animal origin that may be mentioned is perhydrosqualene.

The triglyceride oils of plant or synthetic origin are preferably chosen from liquid fatty acid triglycerides comprising from 6 to 30 carbon atoms, for instance heptanoic or octanoic acid triglycerides, or alternatively, for example, sunflower oil, corn oil, soybean oil, marrow oil, grapeseed oil, sesame seed oil, hazelnut oil, apricot oil, macadamia oil, arara oil, castor oil, avocado oil, caprylic/capric acid triglycerides, for instance those sold by the company Stearineries Dubois or those sold under the names Miglyol® 810, 812 and 818 by the company Dynamit Nobel, jojoba oil and shea butter oil.

The linear or branched hydrocarbons of mineral or synthetic origin containing more than 16 carbon atoms are preferably chosen from liquid paraffins, petroleum jelly, liquid petroleum jelly, polydecenes and hydrogenated polyisobutene such as Parleam®.

The fluoro oils may be chosen from perfluoromethylcyclopentane and perfluoro-1,3-dimethylcyclohexane, sold under the names Flutec® PC1 and Flutec® PC3 by the company BNFL Fluorochemicals; perfluoro-1,2-dimethylcyclobutane; perfluoroalkanes such as dodecafluoropentane and tetradecafluorohexane, sold under the names PF 5050® and PF 5060® by the company 3M, or bromoperfluorooctyl sold under the name Foralkyl® by the company Atochem; nonafluoromethoxybutane and nonafluoroethoxyisobutane; perfluoromorpholine derivatives such as 4-trifluoromethyl perfluoromorpholine sold under the name PF 5052® by the company 3M.

The fatty alcohols that may be used in the cosmetic composition of step a) are saturated or unsaturated, linear or branched alcohols comprising from 6 to 30 carbon atoms and more particularly from 8 to 30 carbon atoms, among which mention may be made, for example, of cetyl alcohol, stearyl alcohol and the mixture thereof (cetylstearyl alcohol or cetearyl alcohol), octyldodecanol, 2-butyloctanol, 2-hexyldecanol, 2-undecylpentadecanol, oleyl alcohol or linoleyl alcohol.

The non-salified fatty acids that may be used in the cosmetic composition of step a) may be saturated or unsaturated carboxylic acids comprising from 6 to 30 carbon atoms and in particular from 9 to 30 carbon atoms. They are more particularly chosen from myristic acid, palmitic acid, stearic acid, behenic acid, oleic acid, linoleic acid, linolenic acid and isostearic acid.

These acids are not salified. This means that they are introduced in the form of free acids and that the composition does not comprise any alkaline agent leading to their salification.

The esters of fatty acids and/or of fatty alcohols, advantageously different from the triglycerides mentioned above, which may be used in the cosmetic composition used in step a) are esters of saturated or unsaturated, linear or branched C1-C26 aliphatic mono- or polyacids and of saturated or unsaturated, linear or branched C1-C26 aliphatic mono- or polyalcohols, the total carbon number of the esters more particularly being greater than or equal to 10. Among the monoesters, mention may be made of dihydroabietyl behenate; octyldodecyl behenate; isocetyl behenate; cetyl lactate; C12-C15 alkyl lactate; isostearyl lactate; lauryl lactate; linoleyl lactate; oleyl lactate; (iso) stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methylacetyl ricinoleate; myristyl stearate; octyl isononanoate; 2-ethylhexyl isononate; octyl palmitate; octyl pelargonate; octyl stearate; octyldodecyl erucate; oleyl erucate; ethyl and isopropyl palmitates, 2-ethylhexyl palmitate, 2-octyldecyl palmitate, alkyl myristates such as isopropyl, butyl, cetyl, 2-octyldodecyl, myristyl or stearyl myristate, hexyl stearate, butyl stearate, isobutyl stearate; dioctyl malate, hexyl laurate, 2-hexyldecyl laurate.

Still within the context of this variant, esters of C4-C22 dicarboxylic or tricarboxylic acids and of C1-C22 alcohols and esters of mono-, di- or tricarboxylic acids and of C2-C26 di-, tri-, tetra- or pentahydroxy alcohols may also be used.

Mention may be made in particular of: diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate, tridecyl erucate; triisopropyl citrate; triisostearyl citrate; glyceryl trilactate; glyceryl trioctanoate; trioctyldodecyl citrate; trioleyl citrate, propylene glycol dioctanoate; neopentyl glycol diheptanoate; diethylene glycol diisononanoate; and polyethylene glycol distearates.

Among the esters mentioned above, it is preferred to use ethyl, isopropyl, myristyl, cetyl or stearyl palmitates, 2-ethylhexyl palmitate, 2-octyldecyl palmitate, alkyl myristates such as isopropyl, butyl, cetyl or 2-octyldodecyl myristate, hexyl stearate, butyl stearate, isobutyl stearate; dioctyl malate, hexyl laurate, 2-hexyldecyl laurate, isononyl isononanoate or cetyl octanoate.

The esters according to this variant may also be chosen from monoesters, diesters, triesters, tetraesters and polyesters, and mixtures thereof. These esters may be, for example, oleates, laurates, palmitates, myristates, behenates, cocoates, stearates, linoleates, linolenates, caprates and arachidonates, or mixtures thereof such as, especially, oleopalmitate, oleostearate and palmitostearate mixed esters. More particularly, use is made of monoesters and diesters and in particular mono- or di-oleate, -stearate, -behenate, -oleopalmitate, -linoleate, -linolenate or-oleostearate of sucrose, glucose or methylglucose.

The non-silicone wax(es) that may be used in the cosmetic composition used in step a) are chosen especially from carnauba wax, candelilla wax, esparto grass wax, hydrocarbon waxes including paraffin wax, ozokerite and microcrystalline wax, plant waxes such as olive wax, rice wax, hydrogenated jojoba wax or the absolute waxes of flowers such as the essential wax of blackcurrant blossom sold by the company Bertin (France), animal waxes, for instance beeswaxes or modified beeswaxes (cerabellina); other waxes or waxy starting materials that may be used according to the invention are especially marine waxes such as the product sold by the company Sophim under the reference M82, and polyethylene waxes or polyolefin waxes in general.

In a preferred embodiment, the one or more non-silicone-based fatty compounds are selected from oils, waxes, linear or branched alkanes, fatty esters, esters of fatty acids, esters of fatty alcohols, cetyl esters, triglycerides, or a mixture thereof.

The total amount of the one or more non-silicone-based fatty compounds in the compositions, if present, will vary. Nonetheless, in various embodiments, the compositions include about 0.1 to about 20 wt. % of the one or more non-silicone-based fatty compounds, based on the total weight of the compositions. In further embodiments, the compositions include about 0.1 to about 15 wt. %, about 0.1 to about 12 wt. %, about 0.1 to about 10 wt. %, about 0.1 to about 8 wt. %, about 0.1 to about 5 wt. %, about 0.5 to about 20 wt. %, about 0.5 to about 15 wt. %, about 0.5 to about 12 wt. %, about 0.5 to about 10 wt. %, about 0.5 to about 8 wt. %, about 0.5 to about 5 wt. %, about 1 to about 20 wt. %, about 1 to about 15 wt. %, about 1 to about 12 wt. %, about 1 to about 10 wt. %, about 1 to about 8 wt. %, about 1 to about 5 wt. %, about 2 to about 20 wt. %, about 2 to about 15 wt. %, about 2 to about 12 wt. %, about 2 to about 10 wt. %, about 2 to about 8 wt. %, about 2 to about 5 wt. %, or about 3 to about 5 wt. %, based on the total weight of the compositions.

### (h) Water-Miscible Solvents

The leave-on finishing composition may further include a water-soluble organic solvent. The term “water-miscible solvent” is interchangeable with the terms “water soluble solvent” and “water-miscible solvent” and “water soluble organic” and “water-miscible organic solvent” and means a compound that is liquid at 25° C. and at atmospheric pressure (760 mmHg), and it has a solubility of at least 50% in water under these conditions. In some cases, the water-soluble solvents has a solubility of at least 60%, 70%, 80%, or 90% in water. Non-limiting examples of water-soluble solvents include, for example, organic solvents selected from glycerin, alcohols (for example C₁₋₈, or C₁₋₄ alcohols), polyols (polyhydric alcohols), glycols, and a mixture thereof.

Nonlimiting examples of water-soluble organic solvents. Non-limiting examples of water-soluble organic solvents include, for example, organic solvents selected from glycerin, alcohols (for example, C₁₋₁₀, C₁₋₈,l or C₁₋₄ alcohols), polyols (polyhydric alcohols), glycols, and a mixture thereof. Nonlimiting examples of monoalcohols and polyols include ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin.

Further non-limiting examples of water soluble organic solvents include alkanediols (polyhydric alcohols) such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and a mixture thereof.

Polyhydric alcohols are useful. Examples of polyhydric alcohols include glycerin, ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a mixture thereof. Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, and a mixture thereof.

Other examples of water miscible solvents include ketal/acetal glycerin compounds. The ketal/acetal glycerin compounds include those of Formula (I):

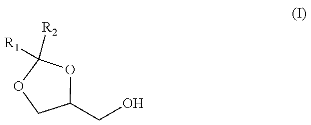

wherein R₁ and R₂ can be the same or different and are independently selected from hydrogen, C₁-C₆ alkyl, C₁-C₆ alkenyl, C₁-C₆ heteroalkyl, C₁-C₆ heteroalkenyl, aryl, heteroaryl, C₃-C₆ cycloalkyl, C₃-C₆ hetero(C₃-C₆) cycloalkyl, aryl, heteroaryl, optionally substituted; or R₁ and R₂ may together form a C₃-C₆ cycloalkyl, C₃-C₆ heterocycloalkyl, aryl, or heteroaryl, optionally, substituted;
wherein heteroatoms are selected from N, O or S;

In certain embodiments, at least one of R₁ and R₂ being a linear, branched, or cyclic C₁-C₆ alkyl. Preferably, R₁ and R₂ are independently a linear C₁-C₆ alkyl, for example isopropylidene glycerol.

In a preferred embodiment, the composition include one or more glycols selected from glycerin, propylene glycol, butylene glycol, pentylene glycol, hexylene glycol, caprylyl glycol, dipropylene glycol, and mixtures thereof.

The total amount of the one or more water soluble solvents in the leave-on finishing composition and/or the conditioning composition, if present, will vary. Nonetheless, in various embodiments, the compositions include about 0.1 to about 20 wt. % of the one or more water soluble solvents, based on the total weight of the compositions. In further embodiments, the compositions include about 0.1 to about 15 wt. %, about 0.1 to about 10 wt. %, about 0.5 to about 20 wt. %, about 0.5 to about 15 wt. %, about 0.5 to about 10 wt. %, about 1 to about 20 wt. %, about 1 to about 15 wt. %, about 1 to about 10 wt. %, about 2 to about 20 wt. %, about 2 to about 15 wt. %, about 2 to about 10 wt. %, about 5 to about 20 wt. %, about 5 to about 15 wt. %, or about 5 to about 10 wt. %, based on the total weight of the compositions. In other embodiments, the one or more water-soluble solvents may be present in a concentration ranging from about 50 to about 98% wt. %, or about 60 to about 98% wt. %.

### (i) Miscellaneous Ingredients

‘The compositions optionally include or exclude (or are essentially free from) one or more miscellaneous ingredients. Miscellaneous ingredients are ingredients that are compatible with the compositions and do not disrupt or materially affect the basic and novel properties of the compositions. Nonlimiting examples of ingredients include preservatives, fragrances, pH adjusters, salts, chelating agents, buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, etc.) composition colorants, etc. In various embodiments, the miscellaneous ingredients are chosen from preservatives, fragrances, pH adjusters, salts, chelating agents, buffers, composition colorants, and mixtures thereof. In the context of the instant disclosure, a “composition colorant” is a compound that colors the composition but does not have an appreciable coloring effect on hair. In other words, the composition colorant is included to provide a coloring to the composition for aesthetic appeal but is not intended to impart coloring properties to hair. Styling gels, for example, can be found in a variety of different colors (e.g., light blue, light pink, etc.) yet application of the styling gel to hair does not visibly change the color of the hair.

The total amount of the one or more miscellaneous ingredients in the compositions, if present, will vary. Nonetheless, in various embodiments, the compositions include about 0.1 to about 15 wt. % of the one or more miscellaneous ingredients, based on the total weight of the compositions. In further embodiments, the compositions include about 0.1 to about 12 wt. %, about 0.1 to about 10 wt. %, about 0.1 to about 5 wt. %, about 0.5 to about 15 wt. %, about 0.5 to about 12 wt. %, about 0.5 to about 10 wt. %, about 0.5 to about 8 wt. %, about 0.5 to about 5 wt. %, about 1 to about 15 wt. %, about 1 to about 12 wt. %, about 1 to about 10 wt. %, about 1 to about 8 wt. %, about 1 to about 5 wt. %, about 2 to about 15 wt. %, about 2 to about 12 wt. %, about 2 to about 10 wt. %, about 2 to about 8 wt. %, or about 2 to about 5 wt. %, based on the total weight of the compositions.

The leave-on finishing composition typically includes:
(a) about 5 to about 20 wt. % of one or more cyclic carbonates;
(b) about 1 to about 8 wt. % of one or more amino-functionalized silicones, wherein (a) and (b) are in a weight ratio of about 12:1 to 1:1 ((a): (b));
(c) about 0.1 to about 4 wt. % of one or more polysaccharides;
(d) about 0.1 to about 3 wt. % of one or more nonionic associative polymeric thickeners;
(e) about 0.5 to about 5 wt. % of one or more nonionic surfactants or emulsifiers;
(f) optionally, one or more non-silicone-based fatty compounds;
(g) optionally, one or more water soluble solvents;
(h) about 60 to about 85 wt. % of water;
   wherein all weight percentages are based on a total weight of the leave-on finishing composition.

The pH of the composition can vary. Nonetheless, in various embodiments, a pH less than 7 (an acidic pH) is desirable. For example, the pH can be from about 3 to about 6.5, about 3 to about 6, about 3 to about 5.5, about 3 to about 5, about 3 to about 4.5, about 3.5 to about 6.5, about 3.5 to about 6, about 3.5 to about 5.5, about 3.5 to about 5, or about 3.5 to about 4.5.

### Exclusions

All components that are positively set forth in the instant disclosure may be negatively excluded from the claims, e.g., a claimed composition may be “free,” “essentially free,” or “substantially free” of one or more components that are positively set forth in the instant disclosure.

In various embodiments, one or more of the hair smoothing composition, the conditioning composition, and/or the leave-on finishing composition (herein after, “hair treatment composition(s)”, is/are free or essentially free from ethylene carbonate. In further embodiments, the hair treatment composition(s) is free or essentially free from carbonates other than the one or more cyclic carbonates. Similarly, in various embodiments, the hair treatment composition(s) is free or essentially free from linear carbonates, e.g., dimethyl carbonate, diethyl carbonate, etc. In various embodiments, the composition is free or essentially free from cyclic lactones (e.g., valerolactone, caprolactone, pantolactone, meadowlactone, etc.), free or essentially free from heterocyclic molecules (e.g., 2-oxazolidinone, 2-imidazolidinone, etc.) free or essentially free from sulfones (dimethyl sulfone, 2,3,4,5-tetrahydrothiophene-1,1-dioxide), and/or free or essentially free from ureas (e.g., urea, ethylene urea, etc.).

In yet further embodiments, the hair treatment composition(s) is free or essentially free from carbonates other than propylene carbonate.

In various embodiments, the leave-on finishing composition in particular is free or essentially free from cationic surfactants. In further embodiments, the hair treatment composition is free or essentially free from cationic conditioning polymers. Similarly, in various embodiments, the composition is free or essentially free from polyquaternium compounds. In further embodiments, the composition is free or essentially free from cationic surfactants and cationic conditioning polymers.

In various embodiments, the hair treatment composition(s) is free or essentially free from anionic surfactants.

In various embodiments, the hair treatment composition(s) is free or essentially free from polymers, copolymers, and crosspolymers formed with acrylate or methacrylate monomers, e.g, free or essentially free from polyacrylic acid and polyacrylate polymers and crosspolymers.

In various embodiments, the hair treatment composition(s) is free or essentially free from N-alkyl-2-mercaptoacetamide. In further embodiments, the hair treatment composition is free or essentially free from all mercaptoacetamides

In various embodiments, the hair treatment composition(s) is free or essentially free from fatty alcohols. In other embodiments, however, the composition includes one or more fatty alcohols, for example, in an amount of about 0.01 to about 10 wt. %, preferably about 0.1 to about 5 wt. %, more preferably in an amount of about 0.5 to about 5 wt. %, based on a total weight of the hair treatment composition(s). Suitable fatty alcohols, if present, include those having a fatty group with a carbon chain of greater than 8 carbon atoms, 8 to 50 carbon atoms, 8 to 40 carbon atoms, 8 to 30 carbon atoms, 8 to 22 carbon atoms, 12 to 22 carbon atoms, or 12 to 18 carbon atoms, including all ranges and subranges therebetween. In some instances, the fatty group of the fatty alcohols has a carbon chain of 10 to 20 carbon atoms or 10 to 18 carbon atoms. The fatty alcohols may be chosen from polyethylene glycol ethers, such as those having a fatty alcohol group with a carbon chain of 12 to 16 or 12 to 14 carbon atoms.

In various embodiments, the hair treatment composition(s) is free or essentially free from silicones other than one or more amino-functionalized silicones. Similarly, in various embodiments, the composition is free or essentially free from silicones other than amodimethicone.

In further embodiments, the hair treatment composition(s) is free or essentially free from monosaccharides and disaccharides. For example, the composition is free or essentially free from ribose, arabinose, glucose, fructose, xylose, sucrose, and/or methyl glucoside.

In various embodiments, the hair treatment composition(s) is free or essentially free from formaldehyde, derivatives of formaldehyde, formalin, and compounds that produce formaldehyde upon heating.

In further embodiments, the hair treatment composition(s) is free or essentially free from thioglycolic acid, thiolactic acid, or salts thereof.

### Embodiments

In various embodiments, the hair smoothing composition comprises or consists of:
(a) about 0.1 to about 20 wt. %, preferably about 0.5 to about 10, more preferably about 2 to about 10 wt. % of one or more keto acids, preferably one or more keto acids are selected from C2-C5 keto acids, more preferably wherein the one or more keto acids includes glyoxylic acid and/or levulinic acid;
(b) about 40 to about 95 wt. % of (water and optional water-miscible solvents),
(c) optionally one or more of the following: non-silicone based fatty compounds, surfactants or emulsifiers, and/or miscellaneous ingredients; and wherein the hair smoothing composition has a pH from about 2.0 to about 5.0.

In various embodiments, the conditioning composition comprises or consists of:
(a) about 0.5 to about 10 wt. % of one or more polar fatty compounds, preferably about 0.5 to about 5 wt. % an such as those having at least 8 caron atoms;
(b) about 50 to about 95 wt. %, of one or more water-miscible solvents, preferably selected from one or more polyols, one or more monoalcohols, and combinations thereof; and
(c) about 0.1 to about 5 wt. %, preferably about 0.5 to about 5 wt. %, more preferably about 0.5 to about 3 wt. % of one or more cationic surfactants, preferably one or more one selected from fatty amidioamines, quaternary amines, and combinations thereof; wherein the conditioning composition is anhydrous.

In various embodiments, the leave-on finishing composition comprises or consists of:
(a) about 1 to about 20 wt. %, preferably about 5 to about 15, more preferably about 8 to about 12 wt. % of one or more cyclic carbonates, preferably one or more cyclic carbonates selected from propylene carbonate, dipropylene carbonate, butylene carbonate, 2,3-butylene carbonate, 2,3-pentylene carbonate, pentylene carbonate, ethylene carbonate, glycerol carbonate, or a mixture thereof, more preferably wherein the one or more cyclic carbonates includes propylene carbonate or is propylene carbonate;
(b) about 1 to about 10 wt. %, preferably about 2 to about 8, more preferably about 3 to about 6 wt. % of one or more amino-functionalized silicones, preferably selected from amodimethicone, bis-hydroxy/methoxy amodimethicone, bis-cetearyl amodimethicone, bis(C13-15 alkoxy) PG amodimethicone, aminopropyl phenyl trimethicone, aminopropyl dimethicone, bis-amino PEG/PPG-41/3 aminoethyl PG-propyl dimethicone, or a mixture thereof, more preferably wherein the one or more amino-functionalized silicones includes amodimethicone or is amodimethicone;
   wherein (a) and (b) are in a weight ratio of about 16:1 to 2:1 ((a):(b)), preferably about 12:1 to about 1:1, more preferably about 8:1 to about 3:1;
(c) about 0.1 to about 5 wt. %, preferably about 0.2 to about 4 wt. %, more preferably about 0.2 to about 3 wt. % of one or more polysaccharides, preferably one or more polysaccharides selected from starches, modified starches, gums, modified gums, celluloses, modified celluloses, and a mixture thereof, more preferably wherein the one or more polysaccharides are selected from xanthan gum, gellan gum, sclerotium gum, guar gum and its derivatives, cellulose and its derivatives, and a mixture thereof, in particular sclerotium gum;
(d) about 0.1 to about 5 wt. %, preferably about 0.2 to about 4 wt. %, more preferably about 0.5 to about 3 wt. % of one or more nonionic associative polymeric thickeners, preferably wherein the one or more nonionic associative polymeric thickeners are selected from polyurethane thickeners, for example, polyurethane/polyether thickeners, more preferably wherein the one or more nonionic associative polymeric thickeners are selected from PEG-240/HDI copolymer bis-decyltetradeceth-20 ether, PEG-150/stearyl alcohol/SMDI copolymer, PEG-150/decyl alcohol/SMDI copolymer, steareth-100/PEG-136/HDI copolymer, or a mixture thereof, in particular, PEG-240/HDI copolymer bis-decyltetradeceth-20 ether;
(e) about 0.2 to about 8 wt. %, preferably about 0.5 to about 6 wt. %, more preferably about 1 to about 5 wt. % of one or more nonionic surfactants or emulsifiers, preferably wherein the one or more nonionic surfactants or emulsifiers are selected from alkoxylated fatty alcohols, fatty acid esters of polyoxyethylene glycol, ethoxylated mono or diglycerides, sorbitan esters, ethoxylated sorbitan esters, fatty acid glycol esters, ethylene oxide, alkyl (ether) phosphates, alkylpolyglucosides, and mixtures thereof, for example, one or more alkyl or polyalkyl ethers of poly(ethylene oxide) containing at least one C8-C30 alkyl radical, with a number of ethylene oxide (EO) units ranging from 3 to 200, in particular, selected from laureth-3, laureth-4, laureth-7, laureth-23, ceteth-5, ceteth-7, ceteth-15, ceteth-23, oleth-5, oleth-7, oleth-10, oleth-12, oleth-20, oleth-50, phytosterol 30 EO, steareth-6, steareth-20, steareth-21, steareth-40, steareth-100, beheneth 100, ceteareth-7, ceteareth-10, ceteareth-15, ceteareth-25, pareth-3, pareth-23, C12-15 pareth-3, C12-13 pareth-4, C12-13 pareth-23, trideceth-3, trideceth-4, trideceth-5, trideceth-6, trideceth-7 and trideceth-10, or mixtures thereof;
(f) optionally, about 0.1 to about 12 wt. %, preferably about 1 to about 10 wt. %, more preferably about 2 to about 8 wt. % of one or more non-silicone-based fatty compounds, preferably wherein the one or more non-silicone-based fatty compounds are selected from oils, waxes, linear or branched alkanes, fatty esters, esters of fatty acids, esters of fatty alcohols, cetyl esters, triglycerides, or a mixture thereof, more preferably wherein the one or more non-silicone-based fatty compounds comprises or consists of one or more linear or branched alkanes, oils, or a mixture thereof;
(g) optionally, about 0.1 to about 15 wt. %, more preferably about 1 to about 12 wt. %, more preferably about 2 to about 10 wt. % of one or more water soluble solvents, preferably wherein the one or more water soluble solvents are selected from glycerin, C₁-C₆ mono-alcohols, polyols (polyhydric alcohols), glycols, or a mixture thereof, more preferably wherein the one or more water soluble solvents are selected from glycerin, glycols (e.g., ethylene glycol, propylene glycol, butylene glycol, pentylene glycol, caprylyl glycol, etc.), or a combination thereof;
(h) about 60 to about 85 wt. %, preferably about 65 to about 80 wt. %, more preferably about 70 to about 80 wt. % of water;
(i) optionally, about 0.01 to about 10 wt. %, preferably about 0.1 to about 8 wt. %, more preferably about 1 to about 5 wt. % of one or more miscellaneous ingredients, preferably selected from preservatives, fragrances, pH adjusters, salts, chelating agents, buffers, antioxidants, flavonoids, vitamins, amino acids, botanical extracts, UV filtering agents, peptides, proteins, protein hydrolysates, and/or isolates, fillers (e.g., organic and/or inorganic fillers such as talc, calcium carbonate, silica, particular materials, etc.), emollients, composition colorants, or a mixture thereof;
   wherein all weight percentages are based on a total weight of the leave-on finishing composition.

### Example 1

### Hair Smoothing Composition

An example Hair Smoothing Composition is shown below in Table 1.

TABLE 1
Hair Smoothing Composition:
HC1

| INCI Name | wt. % |
|---|---|
| GLYOXYLIC ACID | 7 |
| LEVULINIC ACID | 2 |
| SODIUM LEVULINATE | |
| GLYCERIN | 5 |
| ISOPROPYL | 8 |
| MYRISTATE | |
| BETAINE | 1 |
| SURFACTANT | 2.5 |
| ALCOHOL | 3.5 |
| CETEARYL ALCOHOL, | 5 |
| STEARETH-2 | |
| POTASSIUM | 2 |
| HYDROXIDE | |
| WATER / AQUA | Q.S. |

### Example 2

### Conditioning Compositions

An example Conditioning Composition is shown below in Table 2. A comparative Conditioning Composition is shown in Table 3.

TABLE 2
Conditioning Composition: Anhydrous with Polar Fatty
Compound, Cationic surfactant and Water-Miscible Solvent
CC1

| INCI Name | wt. % |
|---|---|
| CETYL ALCOHOL | 2 |
| ISOPROPYLIDENE | 20 |
| GLYCEROL | |
| STEARAMIDOPROPYL | 1.2 |
| DIMETHYLAMINE | |
| OLEIC ACID | 1 |
| PROPYLENE GLYCOL | Q.S. |

TABLE 3
Conditioning Composition: Comparative
CC2

| | | |
|---|---|---|
| (c) | BEHENTRIMONIUM CHLORIDE | 2.4 |
| | CETRIMONIUM CHLORIDE | 0.05 |
| (d) | TRIDECETH-5 | 0.9 |
| | TRIDECETH-10 | 0.2 |
| | TRIDECETH-6 | 0.2 |
| | GLYCERYL LINOLENATE | 0.002 |
| | GLYCERYL OLEATE | 0.04 |
| | GLYCERYL LINOLEATE | 0.06 |
| | SAFFLOWER GLUCOSIDE | 0.01 |
| (e) | CETEARYL ALCOHOL | 6 |
| (f) | AMODIMETHICONE | 4.8 |
| (g) | GLYCERIN | 2.5 |
| | BUTYLENE GLYCOL | 0.1 |
| | CAPRYLYL GLYCOL | 0.3 |
| | ISOPROPYL ALCOHOL | 0.5 |
| (i) | CETYL ESTERS | 1.5 |
| | HELIANTHUS ANNUUS | 0.01 |
| | (SUNFLOWER) SEED OIL | |
| (j) | HYDROXYPROPYLTRIMONIUM | 0.1 |
| | HYDROLYZED WHEAT | |
| | PROTEIN | |
| (k) | MISCELLANEOUS* | ≤5 |
| | (Fillers, pH adjusters, | |
| | preservatives, botanical extract, | |
| | fragrances, buffers, stabilizers, | |
| | vitamins, and/or emollients, etc.) | |
| (h) | WATER | 75 |

### Example 3

### Leave-On Finishing Compositions

Four Leave-On Finishing Compositions are shown below in Table 4.

TABLE 4

| | | Inventive | Comparative | | |
|---|---|---|---|---|---|
| | | LOF1 | LOF2 | LOF3 | LOF4 |
| (a) | PROPYLENE CARBONATE | 10 | 10 | | |
| (b) | AMODIMETHICONE | 2.8 | | 2.8 | |
| Ratio of (a):(b) | | 3.6:1 | | | |
| (c) | SCLEROTIUM GUM | 0.5 | 0.5 | 0.5 | 0.5 |
| (d) | PEG-240/HDI COPOLYMER | 0.9 | 0.9 | 0.9 | 0.9 |
| | BIS-DECYLTETRADECETH- | | | | |
| | 20 ETHER | | | | |
| (e) | TRIDECETH-10 | 0.2 | 0.2 | 0.2 | 0.2 |
| | TRIDECETH-5 | 1.1 | 1.1 | 1.1 | 1.1 |
| (f) | ISONONYL ISONONANOATE | 3 | 3 | 3 | 3 |
| | UNDECANE | 0.7 | 0.7 | 0.7 | 0.7 |
| | TRIDECANE | 0.3 | 0.3 | 0.3 | 0.3 |
| | HELIANTHUS ANNUUS | 0.00003 | 0.00003 | 0.00003 | 0.00003 |
| | (SUNFLOWER) SEED OIL | | | | |
| (g) | BUTYLENE GLYCOL | 5.5 | 5.5 | 5.5 | 5.5 |
| | CAPRYLYL GLYCOL | 0.7 | 0.7 | 0.7 | 0.7 |
| | GLYCERIN | 0.7 | 0.7 | 0.7 | 0.7 |
| (i) | MISCELLANEOUS* | ≤5 | ≤5 | ≤5 | ≤5 |
| | (Preservatives, fragrances, | | | | |
| | buffers, pH adjusters, | | | | |
| | composition colorants, plant | | | | |
| | extracts, emollients, fillers, | | | | |
| | humectants, and/or vitamins, | | | | |
| | etc.) | | | | |
| (h) | WATER | qs | qs | qs | qs |

| pH | 4.5-5.5 | 4.5-5.5 | 4.5-5.5 |
|---|---|---|---|
| Viscosity (25° C.) | 1,000- | 1,400- | 1,400- |
| | 1,300 cSt | 1,600 cSt | 1,600 cSt |

*phenoxyethanol, tocopherol, sodium citrate, acetic acid, sodium hydroxide, potassium laurate

### Example 4

### Testing

Five different routines were evaluated for their effects on cosmetic attributes of hair. The routines were carried out using medium bleached curly hair swatches (with Mizani curl pattern, CP 4). The swatches were initially cleansed with a conventional sulfated shampoo (containing water, surfactants and other ingredients typically found in shampoos) to normalize all the swatches before each routine. After cleansing the hair swatches, they were rinsed, allowed to dry and subjected to the following routines:

The specific compositions applied to the hair are shown in the table below:

TABLE 5

| | | Hair | | Leave-On |
|---|---|---|---|---|
| | | Smoothing | Conditioning | Finishing |
| Routine | Shampoo | Composition | Composition | Composition |
| A | Conventional | — | — | — |
| (Comparative) | shampoo | | | |
| B | Conventional | HS1 | CC2 | LOF4 |
| (Comparative) | shampoo | | | |
| C | Conventional | HS1 | CC1 | LOF2 |
| (Comparative) | shampoo | | | |
| D | Conventional | HS1 | CC1 | LOF3 |
| (Comparative) | shampoo | | | |
| E | Conventional | HS1 | CC1 | LOF1 |
| (Inventive) | shampoo | | | |

1. For all five routines, except Routine A, the hair swatches were treated with a hair smoothing composition HC1 described above in Example 1. Keto acids such as glyoxylic acid provide semi-permanent hair smoothing without breaking the cystine disulfide bridge. It provides long lasting relaxing effects to hair fibers, without causing damage to the hair and scalp irritations. The hair smoothing composition was applied to damp hair swatches, massaged into the hair fibers, and allowed to remain on the hair for 10 minutes. After 10 minutes, the hair smoothing composition was rinsed from the hair swatches. The content of the hair smoothing composition is shown in the table below.
2. Again, except Routine A, the hair swatches were subsequently treated with a conditioning composition that deposits a conditioning film onto the hair fiber. Depending on the routine, the conditioning composition was either CC1 or CC2, as shown in Table 5. The conditioning composition was applied to damp hair swatches, massaged into the hair swatches, and immediately rinsed from the hair and towel dried.
3. The hair swatches were subsequently treated with one of the leave-on finishing compositions of Table 4 (LOF 1,2,3 or 4). The leave-on finishing compositions were individually massaged into hair swatches and allowed to remain on the hair. These compositions were not rinsed from the hair.
4. After application of the leave-on finishing compositions, the hair swatches were blow dried to 100% dry and treated with flat iron at a temperature of 200° C. The hot iron was passed over the hair swatches three times.
5. The hair swatches were subsequently washed with a standard shampoo and conditioned with a standard conditioner. The shampoo-conditioner cycle was carried out 12 consecutive times.

The hair swatches subjected to the above routine were compared with one another. Untreated swatches were used as a baseline. As a control, hair swatches were blow dried and treated with a hot iron at a temperature of 200° C. (3 passes) but not subjected to treatment with the hair smoothing composition, and conditioning rinse (Routine ‘A’). The control swatches were simply cleansed with the standard shampoo, blow dried, and treated with the hot iron.

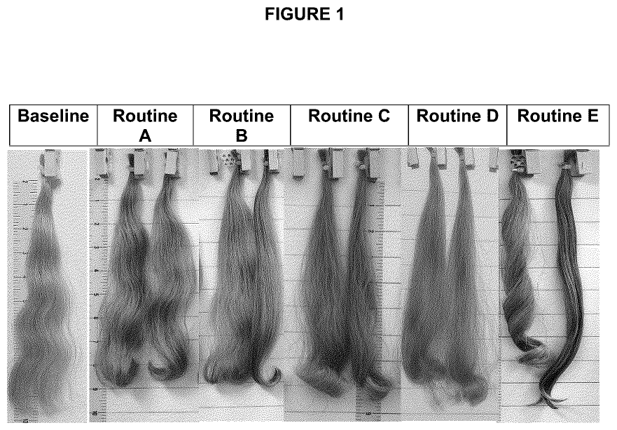

The swatches treated with Routine E showed by far the best fiber alignment, lowest degree of frizz (best frizz control), and the greatest smoothness, as illustrated in The FIG. 1. When the leave-on finishing composition lacks silicone (Comparative Routine C) or lacks propylene carbonate (Comparative Routine B), the degree of alignment, frizz reduction, and smoothness were significantly and surprisingly less.

The foregoing description illustrates and describes the disclosure. Additionally, the disclosure shows and describes only the preferred embodiments. However, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

As used herein, the terms “comprising,” “having,” and “including” are used in their open, non-limiting sense.

The terms “a,” “an,” and “the” are understood to encompass the plural as well as the singular. Thus, the term “a mixture thereof” also relates to “mixtures thereof.” Throughout the disclosure, the term “a mixture thereof” is used, following a list of elements as shown in the following example where letters A-F represent the elements: “one or more elements selected from the group consisting of A, B, C, D, E, F, and a mixture thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counterion. This list of counterions, however, is non-limiting. Appropriate counterions for the components described herein are known in the art.

The expression “one or more” means “at least one” and thus includes individual components as well as mixtures/combinations.

The term “plurality” means “more than one” or “two or more.”

Other than in the operating examples, or where otherwise indicated, all numbers expressing quantities of ingredients and/or reaction conditions may be modified in all instances by the term “about,” meaning within +/−5% of the indicated number.

All percentages, parts and ratios herein are based upon the total weight of the compositions of the present invention, unless otherwise indicated.

Some of the various categories of components identified may overlap. In such cases where overlap may exist and the composition includes both components (or the composition includes more than two components that overlap), an overlapping compound does not represent more than one component. For example, certain compounds may be considered both a nonionic surfactant or emulsifier and a fatty compound. If a particular composition includes both a nonionic surfactant or emulsifier and a fatty compound, a single compound will serve as only the nonionic surfactant or emulsifier or only as the fatty compound (the single compound does not simultaneously serve as both the nonionic surfactant or emulsifier and the fatty component).

A “rinse-off” product refers to a composition that is rinsed and/or washed from the hair with water either after or during the application of the composition onto the hair, and before drying and/or styling the hair. At least a portion of the composition is removed from the hair during the rinsing and/or washing.

A “leave-on” product refers to a composition that is not rinsed and/or washed from the hair after or during application of the composition onto the hair. The composition remains on the hair during drying and/or throughout styling.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range, etc.

The composition of the instant case optionally include one or more surfactants and/or emulsifiers, for example, one or more nonionic, anionic, cationic, and/or amphoteric/zwitterionic surfactants. The term “surfactants” and “emulsifiers” include salts of the surfactants and emulsifiers even if not explicitly stated. In other words, whenever the disclosure refers to a surfactant or emulsifier, it is intended that salts are also encompassed to the extent such salts exist, even though the specification may not specifically refer to a salt (or may not refer to a salt in every instance throughout the disclosure), for example, by using language such as “a salt thereof” or “salts thereof.” Sodium and potassium are common cations that form salts with surfactants and emulsifiers. However, additional cations such as ammonium ions, or alkanolammonium ions such as monoethanolammonium or triethanolammonium ions, may also form salts of surfactants.

The term “substantially free” or “essentially free” as used herein means that there is less than about 2% by weight of a specific material added to a composition, based on the total weight of the compositions. Nonetheless, according to certain embodiments “substantially free” or “essentially free” may refer to less than about 1 wt. %, less than about 0.5 wt. %, less than about 0.1 wt. %, or none of the specified material. “Anhydrous” refers to being substantially free or essentially free of water.

All components that are positively set forth in the instant disclosure may be negatively excluded from the claims, e.g., a claimed composition may be “free,” “essentially free” (or “substantially free”) of one or more components that are positively set forth in the instant disclosure.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. A method for treating hair comprising:
(i) applying a hair smoothing composition comprising (a) at least one keto acid to the hair, allowing the composition to remain on the hair, and rinsing the hair smoothing composition from the hair;
(ii) applying a conditioning composition to the hair, allowing the conditioning composition to remain on the hair, and rinsing the conditioning composition from the hair; the conditioning composition comprising:
(a) one or more polar fatty compounds;
(b) one or more water-miscible solvents; and
(c) one or more cationic surfactants; wherein the conditioning composition is substantially free of water; and
(iii) applying a leave-on finishing composition to the hair; the leave-on finishing composition comprising:
(a) one or more cyclic carbonates;
(b) one or more aminosilicones; and
(c) water.

2. The method of claim 1, wherein the hair smoothing composition remains on the hair for about 1 minute to about 30 minutes before being rinsed from the hair.

3. The method of claim 1, wherein the leave-on finishing composition has a pH of about 2 to about 6, preferably about 2 to about 5.

4. The method of claim 1, wherein the leave-on finishing composition further comprises
(d) a nonionic associative polymeric thickener.

5. The method of claim 1, wherein the leave-on finishing composition further comprises
(e) one or more polysaccharides.

6. The method of claim 1, further comprising:
(iv) after applying the leave-on finishing composition to the hair, drying the hair and styling the hair wherein the styling comprises contacting the hair with a hot iron that is has a temperature of about 150° C. to about 240° C.

7. The method of claim 1, wherein the keto acid is selected from glyoxylic acid, levulinic acid, and combinations thereof.

8. The method of claim 1, wherein the hair smoothing composition further comprises:
(b) about 2 to about 20 wt. % of one or more non-silicone-based fatty compounds comprising:
(b)(i) about 2 to about 12 wt. % of one or more fatty alcohols; and
(b)(ii) optionally, about 1 to about 10 wt. % of one or more additional fatty compounds;
wherein all weight percentages are based on a total weight of the hair smoothing composition.

9. The method of claim 1, wherein the hair smoothing composition further comprises:
(c) about 0.1 to about 5 wt. % of one or more nonionic surfactants or emulsifiers; wherein the weight percentages are based on a total weight of the hair smoothing composition.

10. The method of claim 1, wherein the hair smoothing composition comprises:
(a) about 2 to about 15 wt. % of one or more keto acids comprising:
(a)(i) about 1 to 10 wt. % of glyoxylic acid; and
(a)(ii) about 1 to about 5 wt. % of levulinic acid; and
(b) about 2 to about 20 wt. % of one or more non-silicone-based fatty compounds comprising:
(b)(i) about 2 to about 12 wt. % of one or more fatty alcohols; and
(b)(ii) optionally, about 1 to about 10 wt. % of one or more additional fatty compounds;
(c) about 0.5 to about 8 wt. % of one or more nonionic surfactants or emulsifiers; and
(d) about 40 to about 80 wt. % of water;
wherein all weight percentages are based on a total weight of the hair smoothing composition.

11. The method of claim 1, wherein the conditioning composition comprises:
(a) about 0.5 to about 5 wt. % one or more fatty alcohols;
(b) about 50 to about 95 wt. % of one or more water-miscible solvents; and
(c) about 0.2 to about 5 wt. % of one or more cationic surfactants;
wherein all weight percentages are based on a total weight of the conditioning composition.

12. The method of claim 1, wherein the leave-on finishing composition comprises:
(a) about 5 to about 20 wt. % of one or more cyclic carbonates;
(b) about 1 to about 10 wt. % of one or more amino-functionalized silicones;
(c) about 60 to about 85 wt. % of water;
(d) about 0.1 to about 5 wt. % of one or more nonionic associative polymeric thickeners;
(e) about 0.1 to about 5 wt. % of one or more polysaccharides; and
(f) about 0.2 to about 10 wt. % of one or more nonionic surfactants or emulsifiers; wherein all weight percentages are based on a total weight of the leave-on finishing composition.

13. A method for treating hair comprising:
(i) applying a hair smoothing composition having pH of about 2.0 to about 4.0 to the hair, allowing the composition to remain on the hair, and rinsing the hair smoothing composition from the hair; the hair smoothing composition comprising:
(a) about 2 to about 15 wt. % of one or more acids comprising:
(a)(i) about 1 to 10 wt. % of glyoxylic acid; and
(a)(ii) about 1 to about 5 wt. % of levulinic acid; and
(b) about 2 to about 20 wt. % of one or more fatty compounds comprising:
(b)(i) about 2 to about 12 wt. % of one or more fatty alcohols; and
(b)(ii) optionally, about 1 to about 10 wt. % of one or more additional fatty compounds;
(c) about 0.5 to about 8 wt. % of one or more nonionic surfactants or emulsifiers; and
(d) about 40 to about 80 wt. % of water;
wherein all weight percentages of (i) are based on a total weight of the hair smoothing composition;
(ii) applying a conditioning composition to the hair, allowing the conditioning composition to remain on the hair for a period of time, and rinsing the conditioning composition from the hair; the conditioning composition comprising:
(a) about 0.5 to about 5 wt. % fatty alcohol;
(b) about 50 to about 95 wt. % water-miscible solvents; and
(c) about 0.2 to about 5 wt. % fatty alcohol; and
(d) optionally about 0.5 to about 5 wt. % of one or cationic surfactants
wherein the conditioning composition is substantially free of water and wherein all weight percentages of (ii) are based on a total weight of the conditioning composition; and
(iii) applying a leave-on finishing composition to the hair; the leave-on hair finishing composition comprising:
(a) about 5 to about 20 wt. % of one or more cyclic carbonates;
(b) about 1 to about 10 wt. % of one or more amino-functionalized silicones;
(c) about 60 to about 85 wt. % of water;
(d) about 0.1 to about 5 wt. % of one or more nonionic associative polymeric thickeners;
(e) about 0.1 to about 5 wt. % of one or more polysaccharides; and
(f) about 0.2 to about 10 wt. % of one or more nonionic surfactants or emulsifiers; wherein all weight percentages of (iii) are based on a total weight of the leave-on finishing composition.

14. The method of claim 13, further comprising:
(iv) after applying the leave-on finishing composition to the hair, drying the hair and styling the hair, wherein the styling comprises contacting the hair with a hot iron that is has a temperature of about 150° C. to about 240° C.

15. The method of claim 14, wherein the leave-on finishing composition is not rinsed from the hair prior to drying the hair and styling the hair.
